(19) United States
(12) Patent Application Publication
(10) Pub. No.: US 2008/0051802 A1
(43) Pub. Date: Feb. 28, 2008

(54) MEDICAL INSTRUMENT

(75) Inventors: Sebastian Schostek, Tubingen (DE); Chi-Nghia Ho, Tubingen (DE); Fabian Rieber, Stuttgart (DE); Marc Oliver Schurr, Tubingen (DE)

Correspondence Address: CALFEE HALTER & GRISWOLD, LLP, 800 SUPERIOR AVENUE, SUITE 1400, CLEVELAND, OH, 44114, US

(73) Assignee: NOVINEON HEALTHCARE TECHNOLOGY PARTNERS GMBH, Tubingen (DE)

(21) Appl. No.: 11/836,340

(22) Filed: Aug. 9, 2007

(30) Foreign Application Priority Data
Aug. 10, 2006 (DE) 102006000399.3

(51) Int. Cl. A61B 17/00

(52) U.S. Cl. 606/108; 600/101

(57) ABSTRACT

An insertion aid for medical instruments includes a shaft having a proximal end, a distal end, and a bending part, and a tension element for actuating the bending part. The bending part includes structures which allow a bending in at least one desired direction different from an extension direction of the shaft. The shaft further comprises an outer tube element and an inner tube element supported in the outer tube element so as to be axially movable. The tension element is hinged to one of the inner and outer tube elements and the bending part is provided at a distal end of the other of the inner and outer tube elements.

### CROSS REFERENCE TO RELATED APPLICATION

The present application claims priority to German Patent Application No. DE 102006000399.3, filed on Aug. 10, 2006, the entire disclosure of which is hereby incorporated by reference, to the extent that it is not conflicting with the present application.

### BACKGROUND

Endoscopy is a procedure used in medicine for visual representation of various interior regions of the human body by using an imaging system which is inserted into the body via artificial or natural access paths. Endoscopic procedures allow access to, for example, the abdominal cavity (laparoscopy), the pelvis (pelviscopy), the joints (arthroscopy), the respiratory tract (bronchoscopy) or the digestive tract (gastrointestinal endoscopy) for visual inspection, diagnostic examinations or surgical interventions. Usually, endoscopic procedures cause much less discomfort to the patient than suitable surgical procedures of open surgery since access is possible through natural orifices, for example, in bronchoscopy, gastrointestinal endoscopy, or artificial access can be provided by relatively small cuts within the range of few millimeters to centimeters, such as in laparoscopy or arthroscopy. Besides, the insertion of endoscopic procedures has provided new diagnostic and therapeutic possibilities by specifically developed instruments. Endoscopic procedures typically include the use of a camera system and the presence of a transparent fluid in the space of intervention such as air and/or nitrogen or carbon dioxide with laparoscopy, bronchoscopy and gastrointestinal endoscopy or water with arthroscopy, by which the volume of the space of intervention is kept open.

Normally, access through small cuts and/or natural orifices of the body drastically restricts the degrees of freedom of the inserted instruments, restricts the sensory feedback to a two-dimensional video image and, consequently, demands very good abstractive and coordinative abilities of the surgeon. Hence, the development of endoscopic procedures often involves the development of specialized instruments that compensate, at least partially, for the technical restrictions resulting from the limitations in access, movement, and sensory feedback, by means of various procedures such as adapted operating possibilities or special functions of the instruments.

With percutaneous endoscopic procedures, where the instruments are inserted into the body through small cuts, such as, for example, in laparoscopy or arthroscopy, the positions of the cuts can be to a large extent freely selected within the anatomical borders so that instruments can approach the place of intervention from diverse angles. In the event of endoluminal endoscopic procedures which make use of a natural access path and which are inserted into a tubular and/or tube-like organ, such as, for example, in gastrointestinal endoscopy and bronchoscopy, instruments are guided to a large extent parallel to the optical axis. Thus, in comparison with percutaneous procedures, the degrees of freedom of the instruments used in endoluminal endoscopic procedures are even further restricted.

Furthermore, in particular in gastrointestinal endoscopy as well as bronchoscopy, flexible instrument systems are used in order to be able to follow the anatomy of branched (such as in the bronchial system) or bent and/or sinuous organs, such as the intestine. Such flexible endoscopes can be longer than 2 meters. The endoscope tip of the flexible endoscope system is typically bendable from outside and has a camera system or an optical system with a following image transmitter. Endoscopes used in practice often include one or two working channels through which flexible instruments such as grasping forceps, biopsy forceps, loops or cutting instruments are led out of the endoscope tip. By alignment of the endoscope tip, the instrument tip can be maneuvered to target tissue under visual control. In such cases, the power transmission to the surgical instruments led out at the tip of the endoscope is highly restricted due to the flexible shaft and the extended length.

The endoluminal endoscopic procedures often used today allow various diagnostic and/or therapeutic procedures by using various specific instruments. In conventional gastrointestinal endoscopy, tissue samples are precisely removed, predunculated polyps are cut off by simple loop resection, bleedings are obliterated or appeased, foreign bodies are removed, and stents are positioned. Especially in the field of gastrointestinal endoscopy, in the past few years, new procedures have been developed to fulfill ever more demanding tasks. Therefore, for example, it is possible by using specific instruments to remove, over large regions of the stomach or large intestine, the upper layer of the mucosa in one piece. In this procedure of endoscopic submucosal dissection, ESD, the mucosa is gradually separated and removed from the layer beneath called submucosa.

Demanding procedures such as ESD clearly show the restrictions existing with the degrees of freedom of the conventional instruments used for these procedures. Necessary alignment of such an instrument is effected by controlling the bending of the flexible endoscope. A turning of the instrument is often difficult due to the length and flexibility of the working channel. Thus, the only real option of controlling these instruments typically involves advancing the instrument through the working channel. A further disadvantage of this procedure is that the instrument's axis is linked to the optical axis of the camera system and, thus, the perspective on the instrument cannot be changed.

In an effort to address the problem, various instrument systems have been developed to use instruments with tips that are controlled in a manner independent of the endoscope. Other solutions include instruments that are guided through working channels to the place of intervention which extend outside the endoscope and whose distal orifices are controllable, shown, for example, in PCT Publication No. WO 2004/064600, and in U.S. Pat. No. 6,352,503, the disclosures of which are fully incorporated herein by reference, to the extent they are not conflicting with the present application. Such systems allow an extension of the degrees of freedom and complicated maneuvers may be carried out in a manner to a large extent independent of the endoscope tip. Furthermore, two or more instruments can cooperate to perform a desired function or procedure. For example, it is possible to hold and tension tissue with one instrument while the other instrument precisely cuts the tissue.

There are numerous different approaches for actuating endoscopic instruments for endoluminal procedures. Conventionally, the substantial element of such developments includes a mechanism for bending the instrument tip. Many of these mechanisms, as a result of their kinematics, do not allow a direct, intuitive mechanical control via a mechanically connected grip. Therefore, computer-aided control systems have to convert the input instructions to control instructions so that an intuitive control of the instrument tip may be possible.

### SUMMARY

According to an inventive aspect of the present application, an insertion aid for medical instruments may be configured to be handled more easily while reducing the associated control measures.

The present application contemplates, in one embodiment, an instrument system comprising at least one bendable instrument, an adjustable grip for manual control of the bendable instrument and an overtube device for accommodating and inserting at least one bendable instrument in/into the human body. The camera system is optionally inserted by means of the overtube device or is attached to the distal end of the overtube device.

In one embodiment of the present application, an insertion aid for medical instruments includes a shaft having a proximal end, a distal end, and a bending part, and a tension element for actuating the bending part. The bending part includes structures which allow a bending in at least one desired direction different from an extension direction of the shaft. The shaft further comprises an outer tube element and an inner tube element supported in the outer tube element so as to be axially movable. The tension element is hinged to one of the inner and outer tube elements and the bending part is provided at a distal end of the other of the inner and outer tube elements.

In another embodiment of the present application, an instrument system comprises at least one shaft-like instrument having a bendable distal end, a grip (which may, but need not, be adapted to the human hand) for manual control of the bendable end of the instrument, and an overtube device or guide tube means for accommodating and inserting the at least one shaft-like instrument into the human body. Additionally, a camera system or a visual device may be provided, which can, optionally, also be inserted through the overtube device, or may alternatively be already attached to a distal end of the overtube device.

In still another embodiment, an instrument, which is bendable in at least one preferred direction in connection with the adapted grip, may allow for an intuitive, direct, manual control of the instrument tip. In one embodiment, an instrument shaft has an axially symmetrical design so that there is no preferred bending direction of the instrument shaft. Thus, a turning of the instrument in a bent state is independent of the adjusted angle of rotation. At a distal end, the depicted overtube device has a cover-like connecting bridge which connects individual channels of the overtube device, into which the instruments and/or the camera system can be inserted, at an end and to which a shaft-like or cable-like actuating element may be attached, by which the rotation and advancing of the distal connecting bridge of the overtube device may be controlled from a proximal or extracorporeal end of the overtube device. The instrument channels may be substantially mechanically decoupled from this element. This may facilitate good control of the overtube device while maintaining high flexibility since, for example, in the event of a bending of the overtube device, there is no compression or stretching of the instrument channels, which allows a simple and gentle insertion of the instrument system into the human body.

### BRIEF DESCRIPTION OF THE DRAWINGS

The invention will be explained more precisely below by referring to exemplary embodiments and the attached drawings, wherein:

FIG. 1 illustrates a schematic view of an insertion instrument;

FIG. 2 illustrates a perspective view of an insertion instrument including lateral recesses

FIGS. 3a-3i illustrate schematic views of insertion instruments including recesses of various shapes;

FIGS. 4a-4e illustrate schematic views of insertion instruments including various configurations of recesses;

FIG. 5 illustrates a perspective view of an insertion instrument including hinged segments;

FIGS. 6a-6d illustrate cross-sectional views of insertion instruments including hinge elements in various positions and configurations;

FIG. 7 illustrates a schematic view of an insertion instrument including segments joined by a bending element;

FIG. 8 illustrates a partial side cross-sectional view of two segments of an insertion instrument joined by a bending element;

FIGS. 9a-9c illustrate cross-sectional views of insertion instruments including bending elements in various positions and configurations;

FIGS. 10a-10d illustrate partial cross-sectional views of insertion instruments including bending elements of various shapes;

FIG. 11a illustrates a partial side view of an insertion instrument including a tension element, the instrument being in an unbent orientation;

FIG. 11b illustrates a partial side view of the insertion instrument of FIG. 11a in a first bent orientation;

FIG. 11c illustrates a partial side view of the insertion instrument of FIG. 11a in a second bent orientation;

FIGS. 12a-12e illustrate partial cross-sectional side views of insertion instruments including various mechanical connections between an outer tube element and a tension element;

FIGS. 13a-13j illustrate partial side views of insertion instruments including various mechanical connections between an inner tube element and a tension element;

FIG. 14 illustrates a partial side cross-sectional view of an insertion instrument including a guiding arrangement for a tension element;

FIGS. 15a-15e illustrate partial cross-sectional views of insertion instruments including various guiding arrangements for tension elements;

FIGS. 16a-16d illustrate partial cross-sectional views of insertion instruments including various additional guiding arrangements for tension elements;

FIG. 17 illustrates a partial side cross-sectional view of an insertion instrument including another guiding arrangement for a tension element;

FIGS. 18a-18d illustrate cross-sectional views of insertion instruments including various configurations of inner and outer tube elements;

FIG. 19 illustrates a schematic side view of an endoscopic system including an insertion instrument and a surgical instrument;

FIGS. 20a-20d illustrate cross-sectional views of endoscopic systems including various configurations of inner tube elements and surgical instruments;

FIG. 21a illustrates a partial side schematic view of an endoscopic system including an insertion instrument and a surgical instrument;

FIG. 21b illustrates a partial side schematic view of another endoscopic system including an insertion instrument and a surgical instrument, the system including a load transmitting member;

FIG. 22 illustrates a partial side schematic view of another endoscopic system including an insertion instrument and a surgical instrument, the system including a grip for manual adjustment of the surgical instrument;

FIG. 23 illustrates a partial side schematic view of another endoscopic system including an insertion instrument and a surgical instrument, the system including a pull rod for manipulating a connecting element;

FIG. 24 illustrates a partial side schematic view of another endoscopic system including an insertion instrument and a surgical instrument, the system including a grip for manual rotation of a connecting element;

FIG. 25 illustrates a partial side schematic view of another endoscopic system including an insertion instrument and a surgical instrument, the system including an inner tube element including a bending element;

FIG. 26 illustrates a partial side schematic view of another endoscopic system including an insertion instrument and a surgical instrument, the system including an overtube device including multiple tube elements;

FIG. 27 illustrates a cross-sectional view of an overtube device including multiple tube elements contained in an outer covering;

FIG. 28 illustrates a partial side schematic view of another endoscopic system including an insertion instrument and a surgical instrument, the system including an operating element for manipulating a cable-like control element;

FIG. 29 illustrates a partial perspective view of an overtube device including multiple tube elements and a camera element;

FIG. 30 illustrates a partial perspective view of an inner tube element including a bending element;

FIG. 31a illustrates a partial side view of an endoscopic system having a mechanical gear arrangement including two spur gears for connecting a force transmitting member with a surgical effector;

FIG. 31b illustrates a partial side view of an endoscopic system having a mechanical gear arrangement including two bevel gears for connecting a force transmitting member with a surgical effector;

FIG. 32 illustrates a partial side view of an endoscopic system having a manual operating element for manipulation of an instrument tip, the manual operating element including grips;

FIG. 33 illustrates a partial side view of an endoscopic system having a manual operating element for manipulation of an instrument tip, the manual operating element including a trigger connected with a force transmitting line;

FIG. 34 illustrates a partial side view of an endoscopic system having a manual operating element for manipulation of an instrument tip, the manual operating element including a compressible spring;

FIG. 35 illustrates a partial side view of an endoscopic system having first and second operating element for manipulation of an instrument tip;

FIG. 36 illustrates a partial side view of another endoscopic system having first and second operating element for manipulation of an instrument tip;

FIG. 37 illustrates a partial side view of an endoscopic system having two surgical devices connected with an instrument tip by two connecting elements;

FIG. 38a illustrates a partial side view of an endoscopic system having an operating element connected with a control element for control of a surgical effector;

FIG. 38b illustrates a partial side view of an endoscopic system having an operating element for rotation of a surgical effector;

FIG. 39 illustrates a partial side view of an endoscopic system having an operating element connected with a surgical effector by a flexible shaft;

FIG. 40 illustrates a partial side view of an overtube device including a control element;

FIG. 41 illustrates a partial side view of another overtube device including a control element;

FIGS. 42a-42d illustrate cross-sectional views of endoscopic systems including various configurations of inner tube elements and surgical devices;

FIGS. 43a-43d illustrate cross-sectional views of endoscopic systems including various configurations of guiding devices, tube elements, and control elements;

FIG. 44 illustrates a partial side perspective view of an endoscopic system including guiding segments connecting a tube element with a control element;

FIG. 45 illustrates a partial side perspective view of an actuating device for an endoscopic system;

FIGS. 46a and 46b illustrate partial end views of a hose element and distal end element for an endoscopic system;

FIG. 47 illustrates a partial side view of an endoscopic system including two control elements connected with a distal end element;

FIG. 48 illustrates a cross-sectional view of an overtube device for an endoscopic system;

FIG. 49 illustrates a partial side perspective view of an overtube device for an endoscopic system;

FIG. 50 illustrates a cross sectional view of an overtube device for an endoscopic system disposed between fluid chambers within a hollow organ;

FIG. 51a illustrates a cross-sectional view of a fluid chamber system for an endoscopic system, the fluid change system being shown in a supported condition; and

FIG. 51b illustrates a cross-sectional view of the fluid chamber system of FIG. 51a, the fluid chamber system being shown in a collapsed condition.

### DETAILED DESCRIPTION

Instrument.

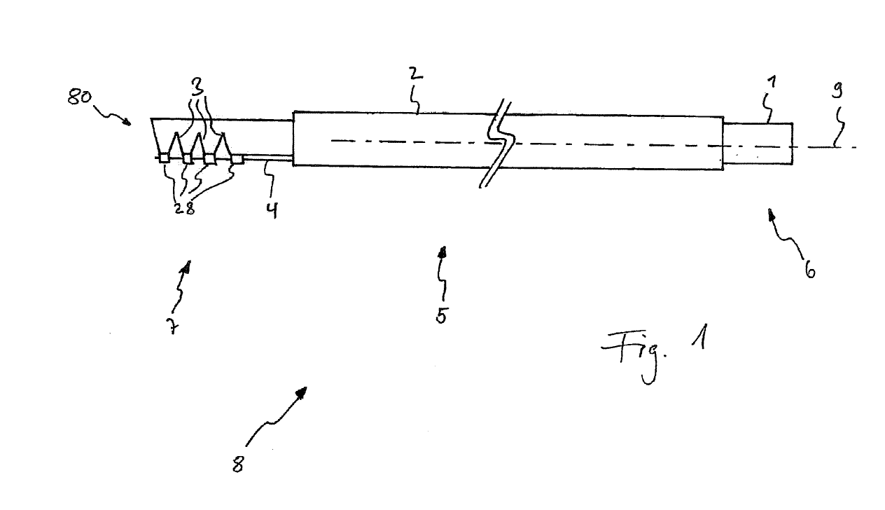

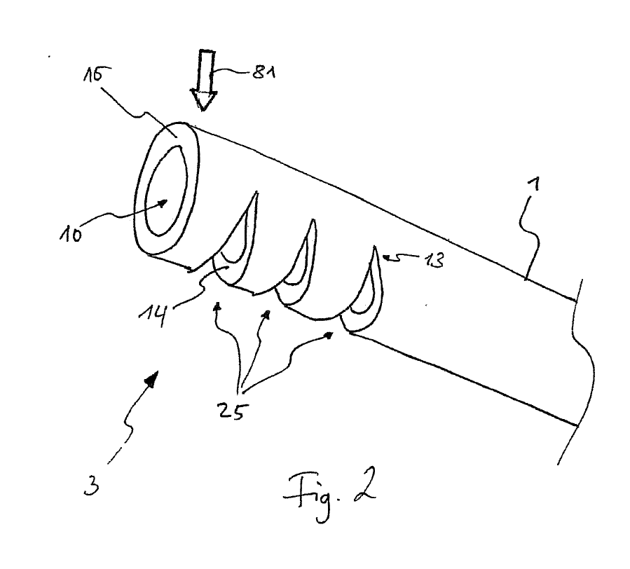

The instrument 8 according to one embodiment of the present application, as shown in FIG. 1, is designed as an endoscope and serves for orientation of a second device 45 (shown, for example, in FIG. 19), such as a surgical instrument, in at least one desired direction 81 (see FIG. 2). The endoscope like instrument 8 comprises an instrument shaft 5 having a distal end 7 and a proximal end 6. The illustrated distal end 7 has hinge-like first structures 3 or target bending points which allow bending of a distal instrument tip 80 in the at least one preferred direction 81. The instrument 8 further has a tension element 4 by actuation of which the bending of the instrument tip 80 can be effected. The instrument shaft 5 has an inner tube element 1 and an outer tube element 2, the inner tube element 1 being guided in the outer tube element 2. The inner tube element 1 protrudes from the outer tube element 2 at the distal end 7 of the instrument 8 and has first structures 3 in the protruding portion. The outer tube element 2 is movable parallel to the instrument axis 9 in relation to the inner tube element 1 and is connected to the tension element 4. When moving the outer tube element 2 parallel to the instrument axis 9 towards the proximal end 6 of the instrument 8, the tension element 4 is actuated, which effects a bending of the instrument tip 80 in the preferred direction 81 within the region of the first structures 3. Furthermore, the tension element 4 may be appropriately configured such that a bending of the instrument tip 80 in a direction opposite to the preferred bending direction can be effected upon moving the outer tube element 2 parallel to the instrument axis 9 towards the distal end 7 of the instrument 8. By an axially symmetrical design of the instrument 8 within the region of the instrument shaft 5, a preferred bending direction of the instrument shaft 5 is avoided. Therefore, upon rotation of the instrument 8 in a bent state, the required torque is independent of the adjusted bending angle.

Referring now to FIG. 2, the illustrated first structures 3 serve for generating a preferred bending direction 81 in a limited section of the inner tube element 1 at the distal end 7 of the instrument 8. This is achieved by clearances or notches 25 in the inner tube element 1 which are arranged at longitudinal distances at the inner tube element 1. Each of the notches 25 has opposing faces 14 approaching each other upon bending of the instrument tip 80. As shown, the first structures 3 may be designed so that the axial bore of the inner tube element 1 is not restricted.

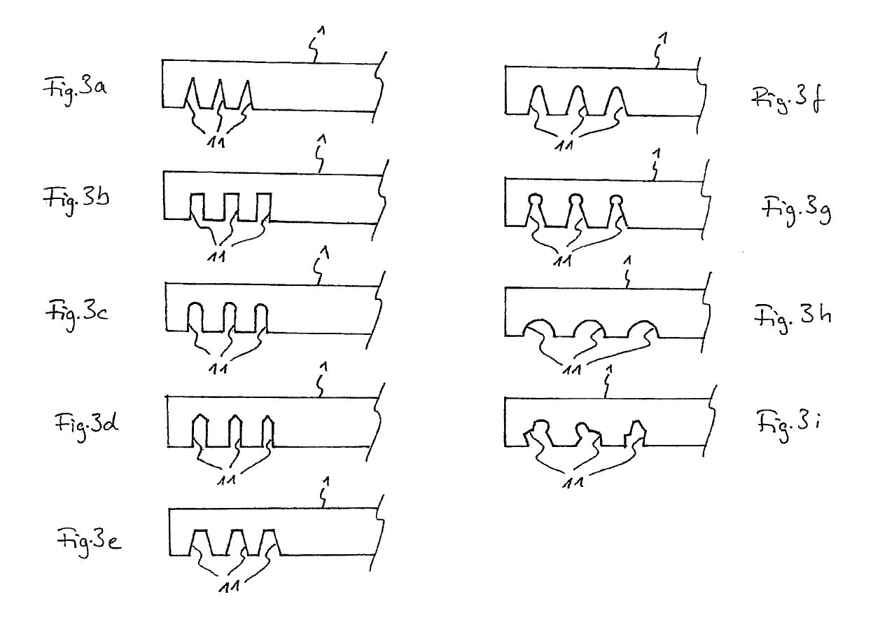

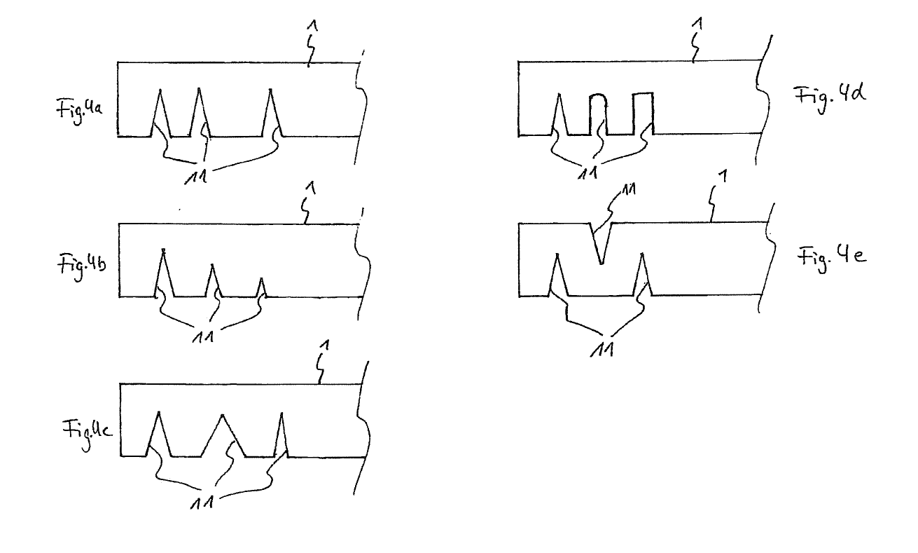

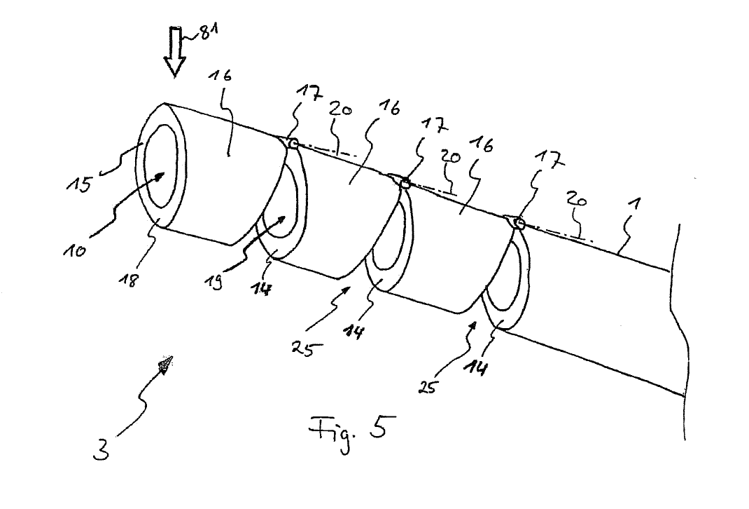

The first structures 3 may be shaped or otherwise configured to form a variety of lateral recesses on the inner tube element 1 at the distal end 7 of the instrument 8. Examples of possible shapes and configurations of these lateral recesses 11 are illustrated in FIGS. 3a-3i.

In one embodiment of the inner tube element 1, the lateral recesses 11 have a triangular cross-section, as shown in FIG. 3a. In another embodiment of inner tube element 1, the lateral recesses 11 have a rectangular cross-section, as shown FIG. 3b. In still another embodiment of the inner tube element 1, the lateral recesses 11 have a cross-section where the faces 14 (see FIG. 2) of the recesses extend in parallel and the bottoms 13 of each recess are rounded, as shown in FIG. 3c. In another embodiment, the lateral recesses 11 may each be provided with parallel faces and a v-shaped bottom, as shown in FIG. 3d. In yet another embodiment, the lateral recesses 11 may be provided with trapezoidal cross-sections, as shown in FIG. 3e. In another embodiment, the lateral recesses 11 may be provided with a triangular cross-section where the bottom of the recess is chamfered or rounded, as shown in FIGS. 3f and 3g. This chamfering may allow for a reduction of local tension excesses within the region of the vertex of the triangle when the instrument tip 80 is bent. In still another embodiment, the lateral recesses 11 may each have a semicircular cross-section, as shown in FIG. 3h. In another embodiment, the lateral recesses 11 may be provided with an irregular cross-section, as shown in FIG. 3i.

While the lateral recesses 11 may be arranged at regular distances from each other, as shown in FIGS. 3a-3i, in another embodiment, the lateral recesses 11 may be arranged at different distances with respect to each other, as shown in FIG. 4a. Further, while each of the lateral recesses 11 may be provided with uniform depth, as shown in FIGS. 3a-3i, in another embodiment, each of the lateral recesses 11 may be provided with a different depth, as shown in FIG. 4b. Further still, while each of the lateral recesses 11 may have the same width, as shown in FIGS. 3a-3h, in another embodiment, each of the lateral recesses 11 may have a different width, as shown in FIG. 4c. Additionally, while each of the lateral recesses may have the same shape, as shown in FIGS. 3a-3h, in another embodiment, each of the lateral recesses 11 has a different shape, as shown in FIGS. 3i and 4d. Still further, while the lateral recesses 11 may be aligned in the same direction (or on the same side of the inner tube element 1), preferably in a desired bending direction, as shown in FIGS. 3a-3i, in another embodiment, the lateral recesses 11 may be aligned in different directions, as shown in FIG. 4e.

A further example of the design of the first structures 3, as shown in FIG. 5 provides a connection of individual segments 16 of the inner tube element 1 through external hinge elements 17. The individual tube segments 16 are preferably arranged so that a continuous chain of segments 16 connected to each other by the hinge elements 17 is obtained. Each of these tube segments 16 can be tilted and/or bent in relation to the adjacent segment 16 around the pivot axis 20 of the hinge element 17 which is disposed between the adjacent segments 16, respectively. Each of the segments 16 has an axial through bore 19.

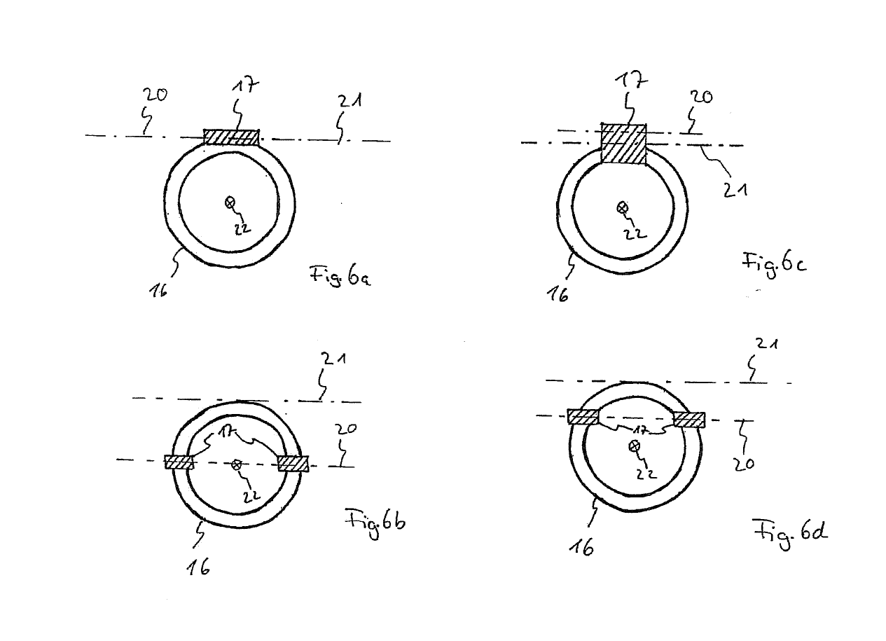

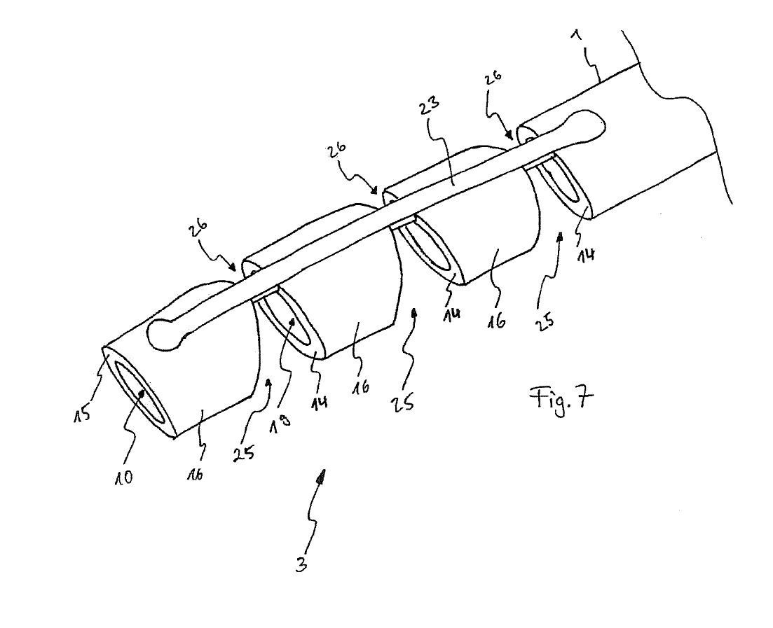

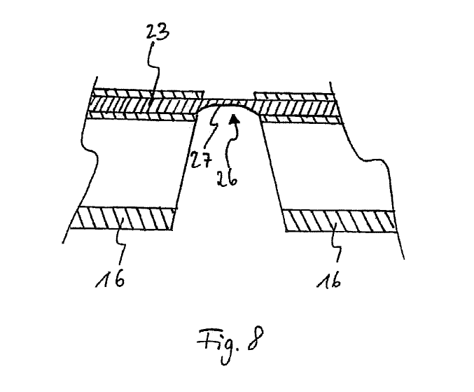

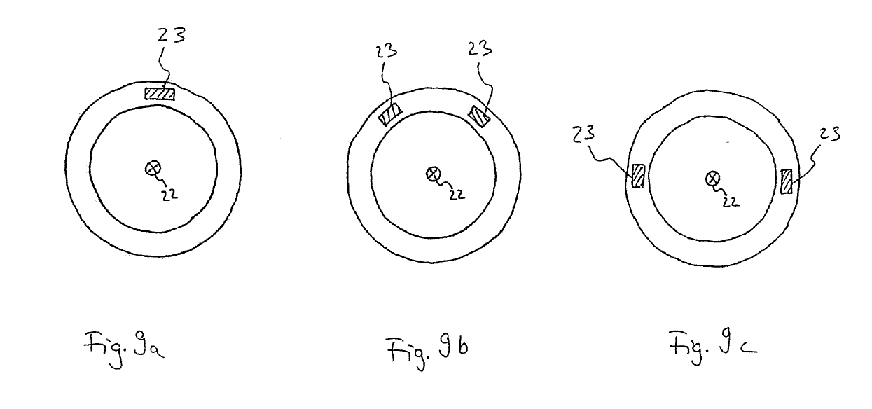

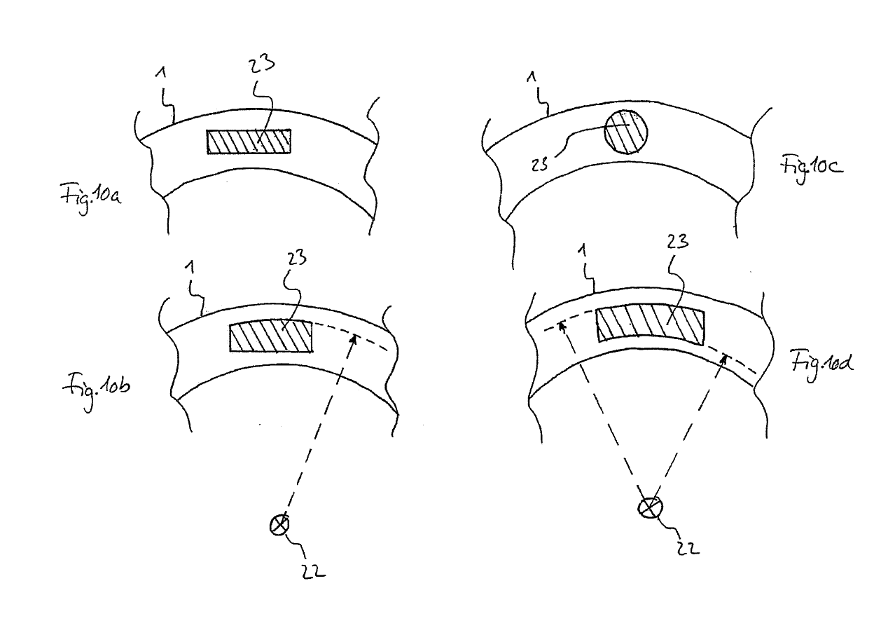

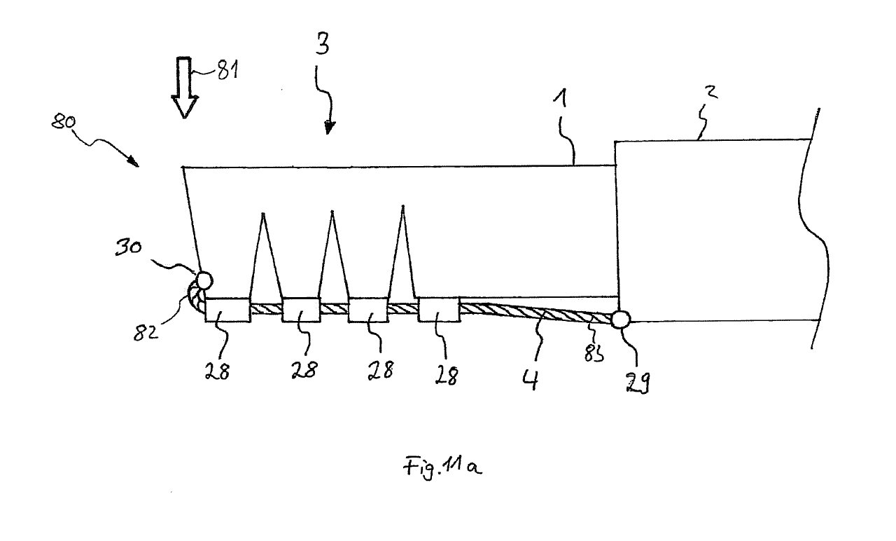

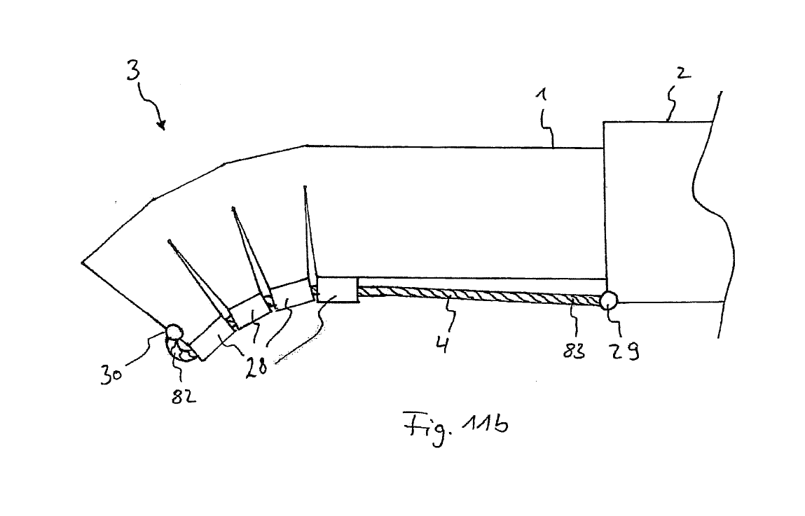

In one embodiment, the hinge element 17 may be connected to a segment 16 so that the pivot axis 20 of the hinge element 17 extends collinearly with a line 21 tangential to the outer surface of the segment, as shown in FIG. 6a. In another embodiment, the hinge element 17 may be connected to a segment 16 so that the pivot axis 20 of the hinge element 17 crosses the axis 22 of the segment 16, as shown in FIG. 6b. In yet another embodiment, the hinge element 17 may be connected to a segment 16 so that the pivot axis 20 of the hinge element 17 extends outside the segment 16, as shown in FIG. 6c. In still another embodiment, the hinge element 17 is connected to a segment 16 so that the pivot axis 20 of the hinge element 17 extends between a line 21 tangential to the outer surface of the segment and the axis 22 of the segment, as shown in FIG. 6d.

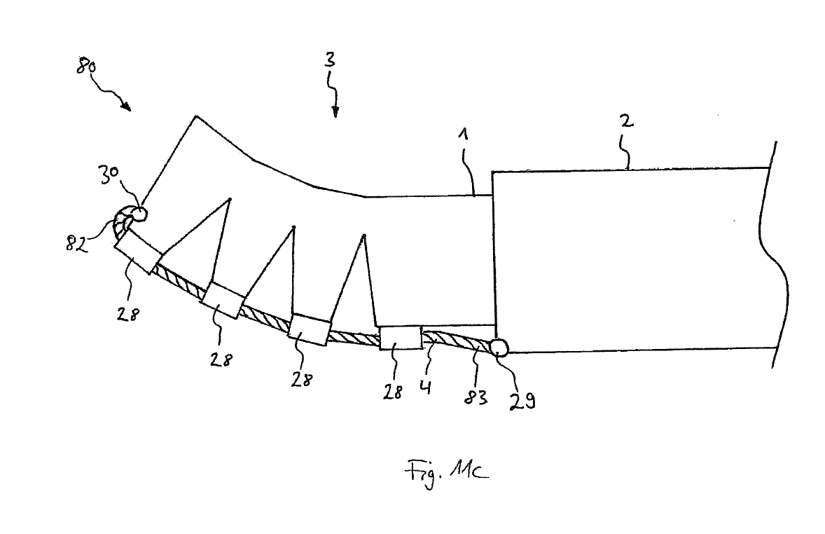

A further example of the design of the first structure 3 involves the connection of individual tube segments 16 through at least one external bending element 23, as shown in FIG. 7. The individual tube segments 16 of the illustrated embodiment are preferably arranged so that a continuous chain of segments 16 connected to each other through the external bending element 23 is obtained. Each of these segments 16 can be tilted in relation to the adjacent segment 16 by deformation of the bending element 23 within the bending region 26. Each of the segments has an axial through bore 19.

The illustrated bending element 23 serves for deformation when the instrument tip 80 is bent. Therefore, there may be less mechanical load on other elements such as the segments 16 than in the event of use of lateral recesses 11 while a deformation-dependent reset force may be more easily realized than in the event of use of hinge elements 17. By a suitable design of the bending element 23, the mechanical properties can be efficiently influenced. As one example, shape memory alloys such as nickel-titanium alloys are suitable as material for the bending element 23, as they are adapted to facilitate a return to the original state even after strong deformation has taken place.

In one such embodiment of the bending element 23, the bending element 23 is made of a shape memory alloy, preferably a nickel-titanium alloy. In another embodiment, as shown in FIG. 8, the bending element 23 may include a narrowed portion 27 within the bending region 26. The narrowed portion 27 localizes the deformation upon bending of the instrument tip to the bending regions 26 of the bending element 23, said bending regions being optionally positioned between the segments 16.

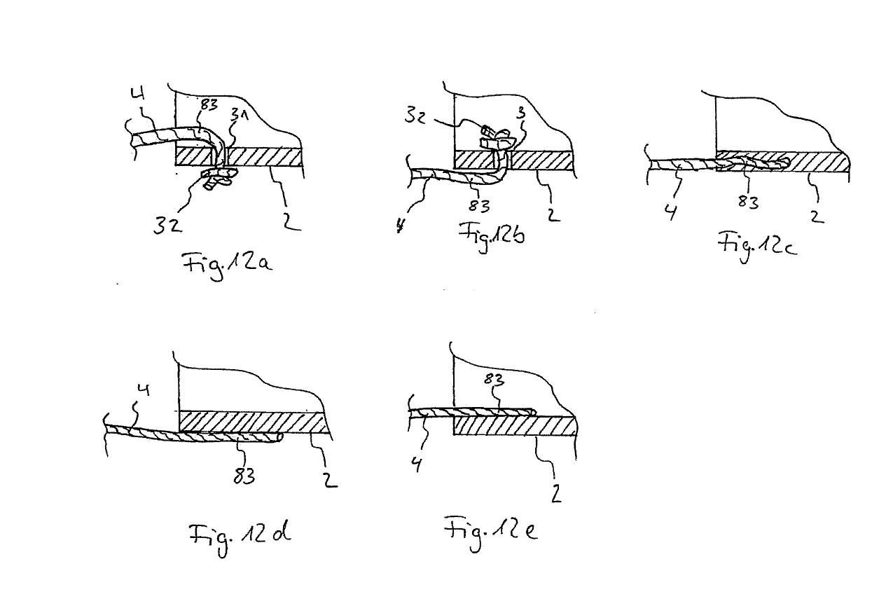

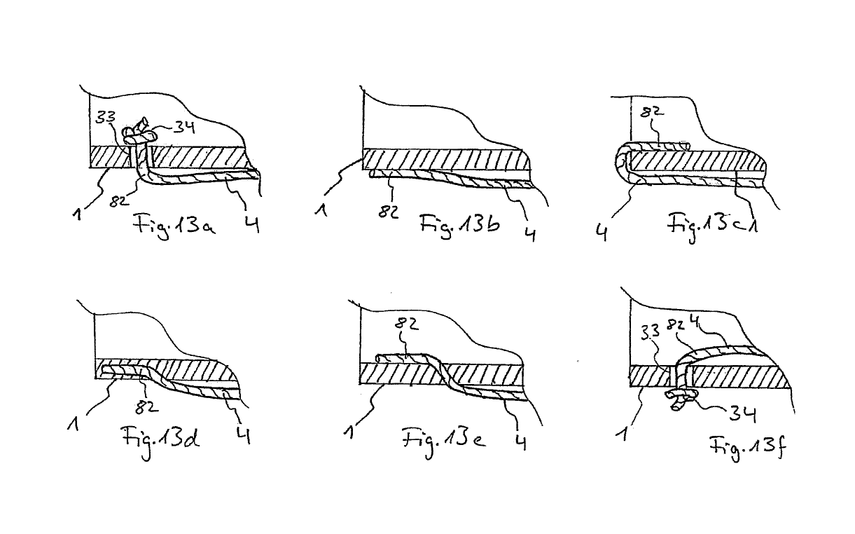

The bending element 23 may be provided in a variety of positions or orientations. In one embodiment, the bending element 23 may include one web in a bending region 26, as shown in FIG. 9a. In another embodiment, the bending element 23 may include two webs in the bending region 26, as shown in FIG. 9b. In still another embodiment, the bending element 23 may include two webs located at diametrically opposite points of the inner tube element 1, as shown in FIG. 9c.

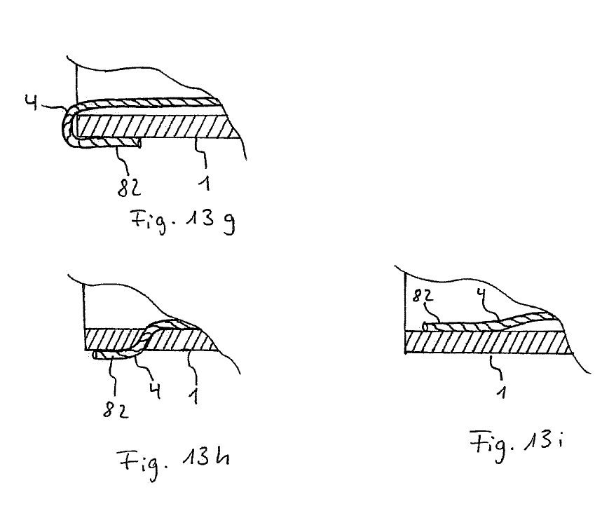

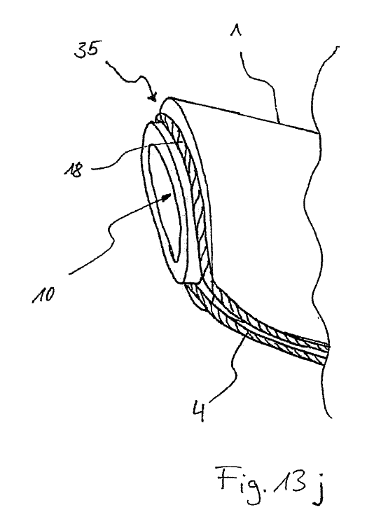

The bending element may be provided with a variety of cross-sectional shapes. In one embodiment, the bending element 23 may include a rectangular cross-section in the bending region 26, as shown in FIG. 10a. In another embodiment, the bending element 23 may include a cross-section in the bending region 26 which is, on the outer surface of the tube segment, designed so as to form a convexity, as shown in FIG. 10b. In yet another embodiment, the bending element 23 may include a circular cross-section in the bending region 26, as shown in FIG. 10c. In still another embodiment, the bending element 23 may include a cross-section in the bending region 26 which forms a convexity on the outer surface side of the tube segment and a concavity on the inner surface side of the tube segment, as shown in FIG. 10d.

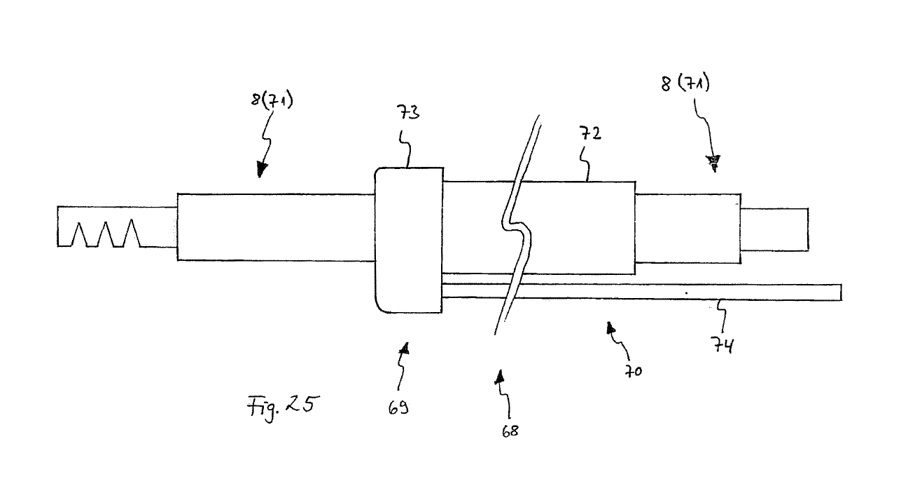

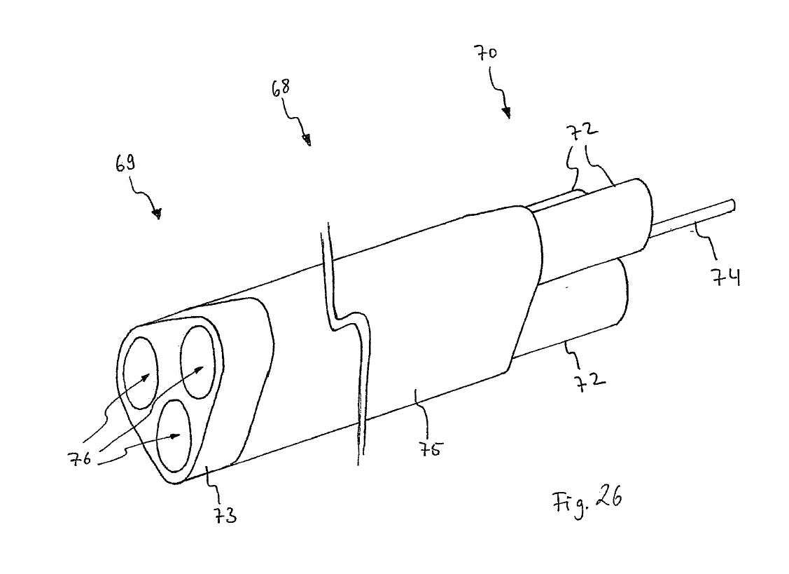

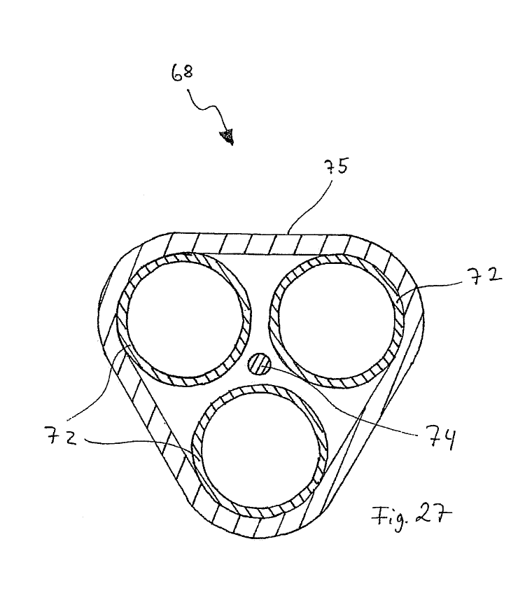

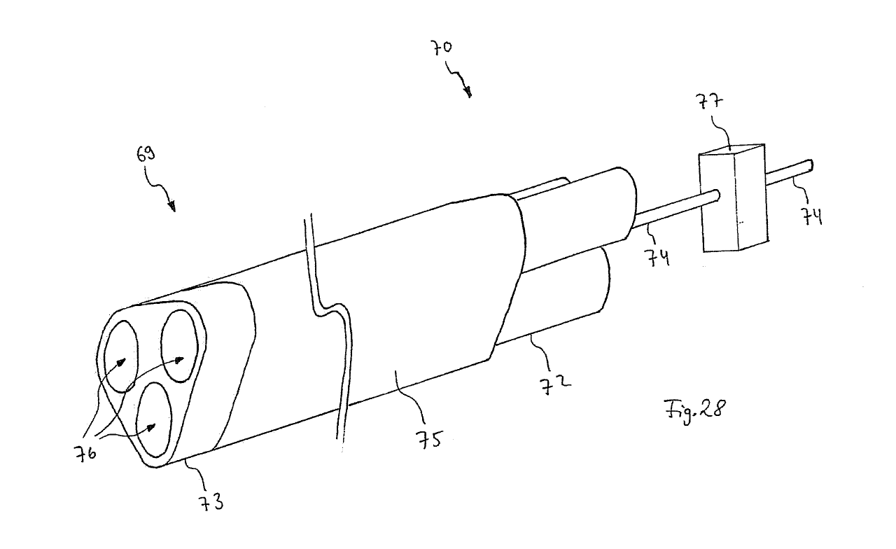

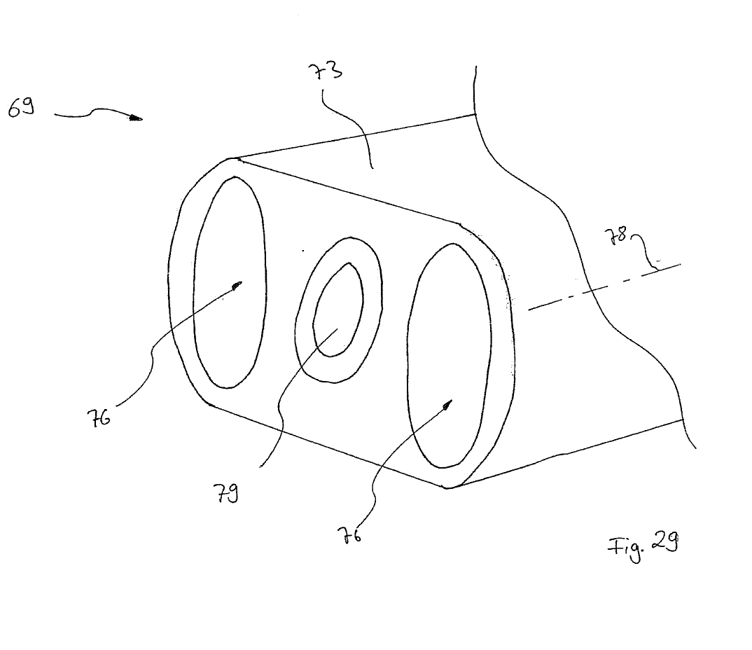

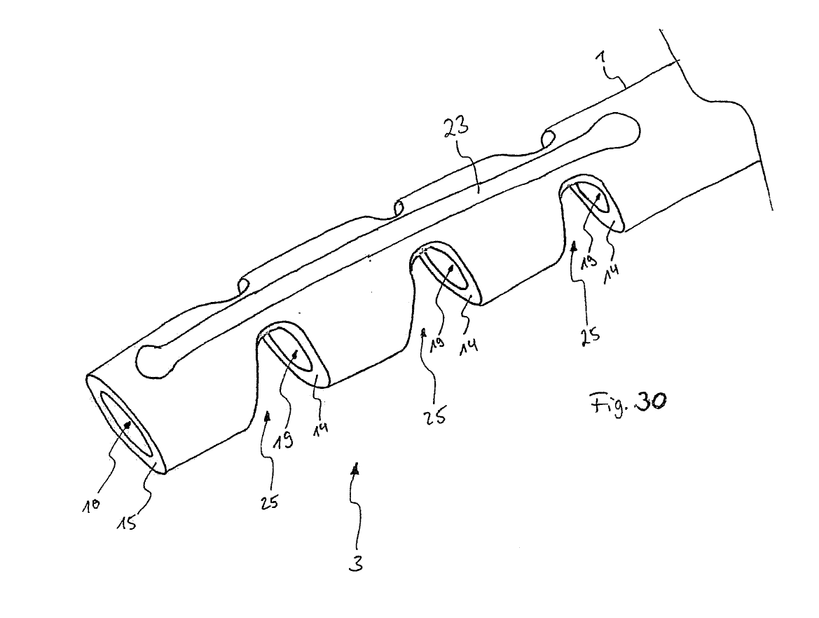

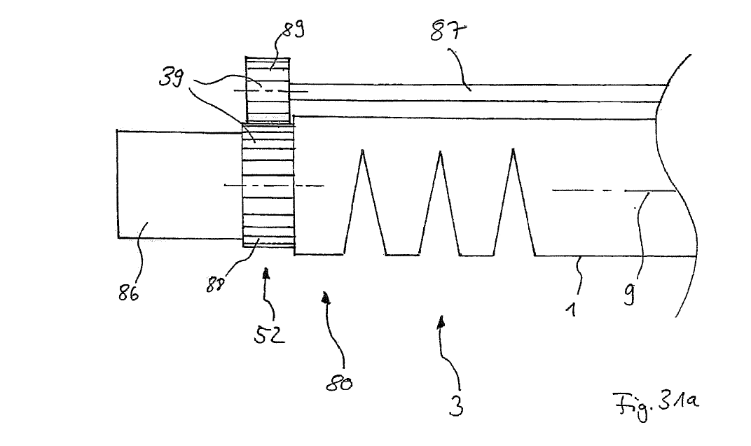

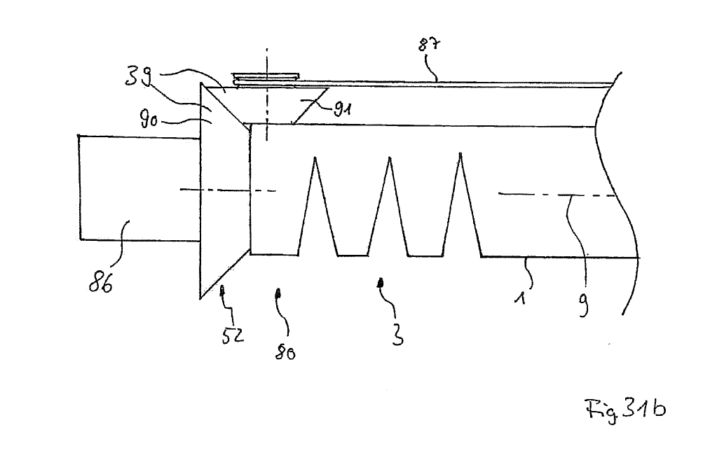

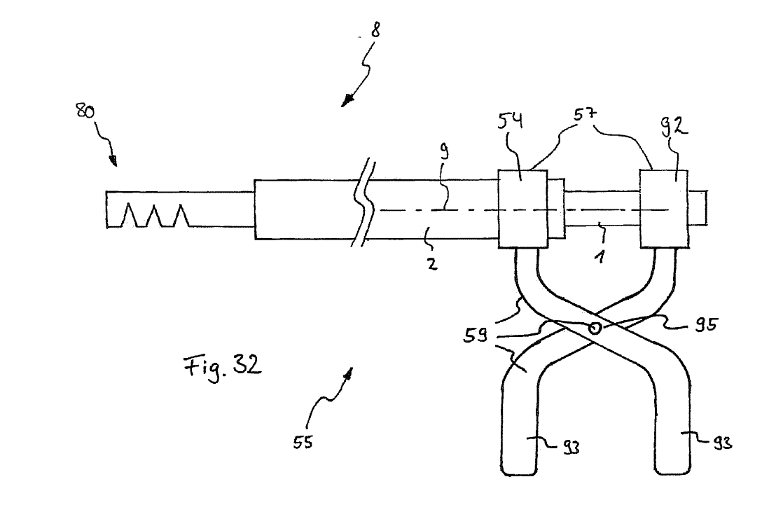

A further example of a design of the first structures 3 relates to a combination of an external bending element 23 including lateral recesses 11 integrated in the inner tube element 1. Thus, first structures 3 can be realized by embedding a bending element 23 into the inner tube element 1 and by providing the lateral recesses 11 with a minimal number of components, and their mechanical properties may be adjusted by a selective design of the bending element 23. For example, such an arrangement may be incorporated into the embodiment of FIG. 25.

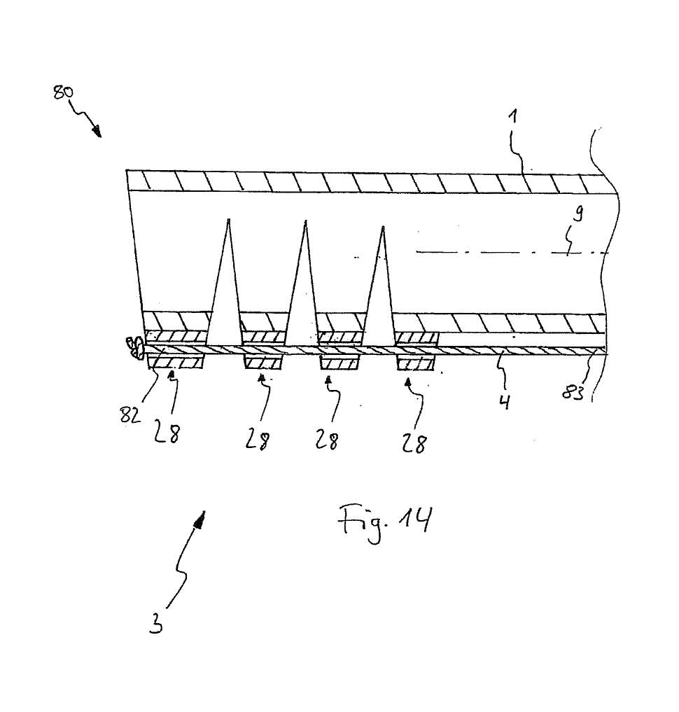

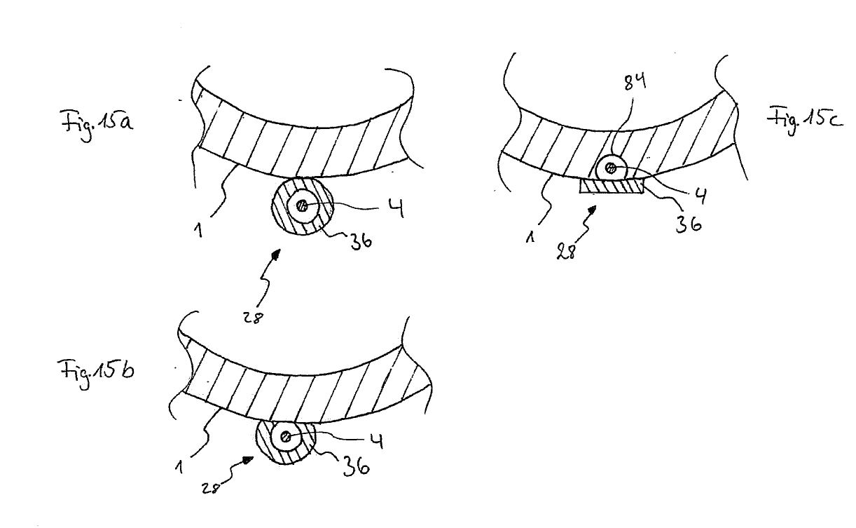

According to another inventive aspect of the present application, a tension element or tension/compression element 4 may serve for load transmission between the outer tube element 2 and the first structures 3. Moreover, the tension element 4 may be connected to the outer tube element 2 via a first mechanical connection 29 and to the instrument tip 80 via a second mechanical connection 30, as shown, for example, in FIGS. 11a-11c. The first structures 3 may be located between the first mechanical connection 29 and the second mechanical connection 30. The tension element 4 may be guided from the first mechanical connection 29 to the second mechanical connection 30 past the first structures 3 on the side of the desired bending direction 81. In doing so, the tension element 4 may be guided by a second tension element guide 28 on the side of the desired bending direction 81, connected to the inner tube element 1 within the region of the first structures 3.

The second tension element guide 28 may be designed so that, upon moving the outer tube element 2 parallel to the instrument axis 9 towards the proximal end 6 of the instrument 8, the instrument tip 80 is bent in the direction of the preferred direction 81, as shown in FIG. 11b. Furthermore, the tension element 4 and the second tension element guide 28 may be optionally designed according to the principle of a Bowden cable such that, upon moving the outer tube element 2 parallel to the instrument axis 9 towards the distal end 7 of the instrument 8, the instrument tip 80 is bent opposite to the preferred direction 81, as shown in FIG. 11c. For doing so, the tension element guide may include a number of eyes or eyelets which are mounted on the individual tube segments in the region between the notches, respectively, such that the eyelets are aligned substantially in a line.

As shown in FIGS. 11a-11c, the first mechanical connection 29 connects the proximal end 83 of the tension element 4 to the outer tube element 2 so that, if a force acts upon the tension element 4 towards the distal end 7 of the instrument 8, the force is transmitted to the outer tube element 2. Optionally, the first mechanical connection 29 connects the proximal end 83 of the tension element 4 to the outer tube element 2 so that, if a force acts upon the tension element towards the proximal end 6 of the instrument 8, the force (compressive force) is transmitted to the outer tube element 2.

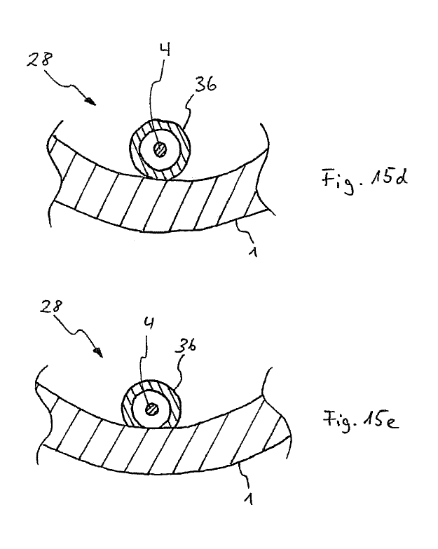

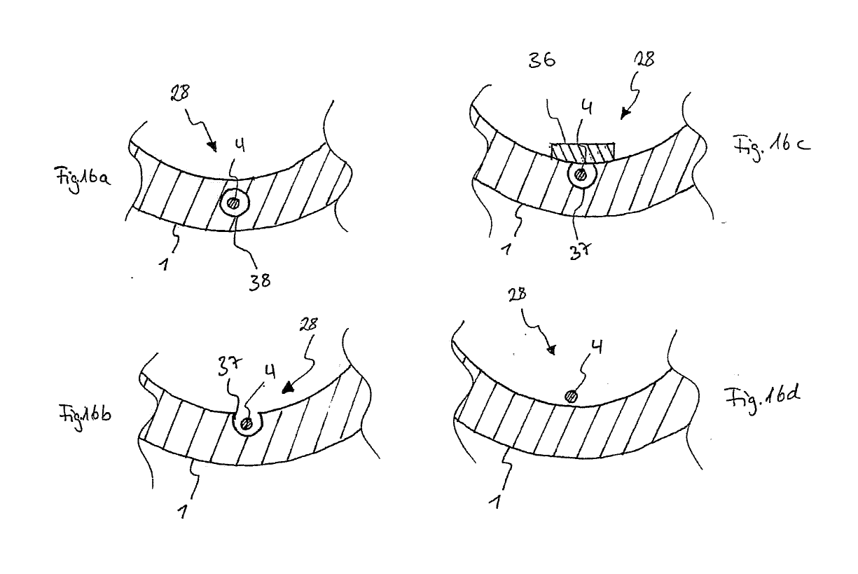

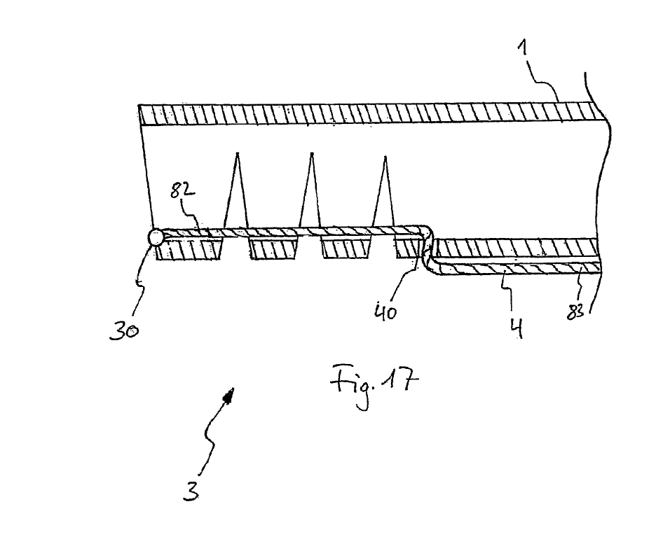

Many different types of mechanical connections 29 between the tension element 4 and the outer tube element 2 may be used. In one embodiment, as shown in FIGS. 12a and 12b, the tension element 4 may be guided through a lateral opening 31 in the outer tube element 2, with the tension element 4 having a proximal enlargement 32, such as, for example, a knob or a component part which is mechanically fixed to the tension element 4 and which blocks a passing of the proximal end 83 of the tension element 4 through the lateral opening 31 in the outer tube element 2. The proximal enlargement may be located on the outer surface of the outer tube element 2, as shown in FIG. 12a, or on the inner surface of the outer tube element 2, as shown in FIG. 12b. In another embodiment, a proximal end 83 of the tension element 4 may be embedded in the wall of the outer tube element 2, as shown in FIG. 12c. In still another embodiment, a proximal end 83 of the tension element 4 may be connected to the outer tube element 2 on the outer surface of the outer tube element 2, as shown in FIG. 12d, or on the inner surface of the outer tube element 2, as shown in FIG. 12e.

As shown in the embodiment of FIGS. 11a-11c, a second mechanical connection 30 may connect the distal end 82 of the tension element 4 to the inner tube element 1 so that, if a force acts upon the tension element 4 towards the proximal end 6 of the instrument 8, the force is transmitted to the inner tube element 1. Optionally, the second mechanical connection 30 connects the distal end 82 of the tension element 4 to the inner tube element 1 so that, if a force acts upon the tension element towards the distal end 7 of the instrument 8, the force is transmitted to the inner tube element 1.

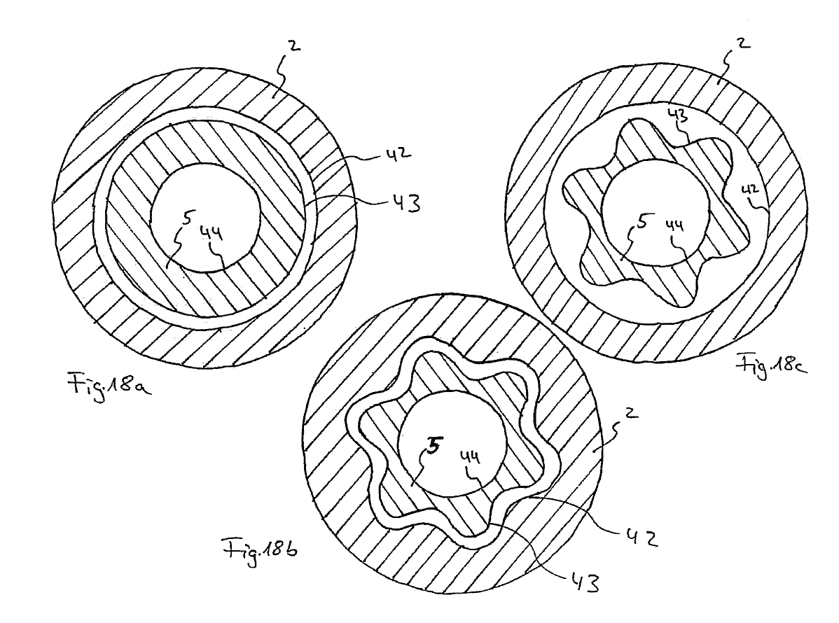

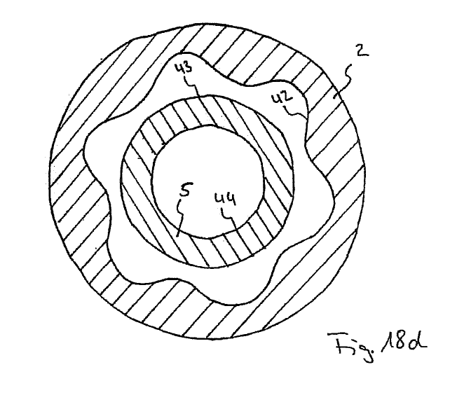

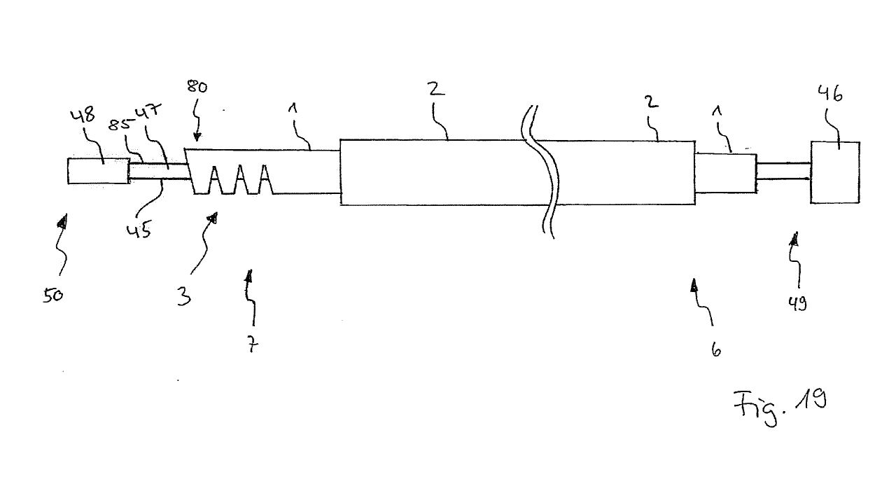

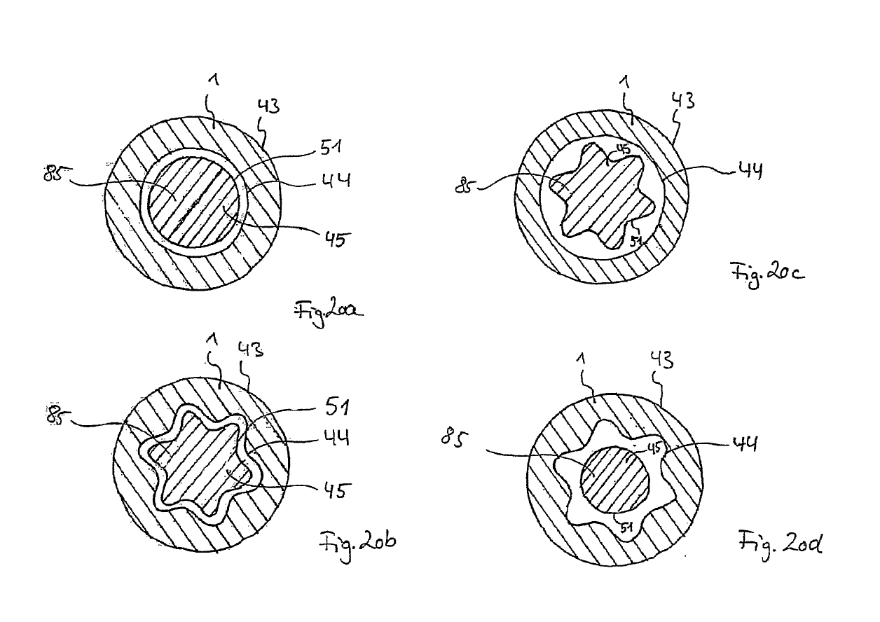

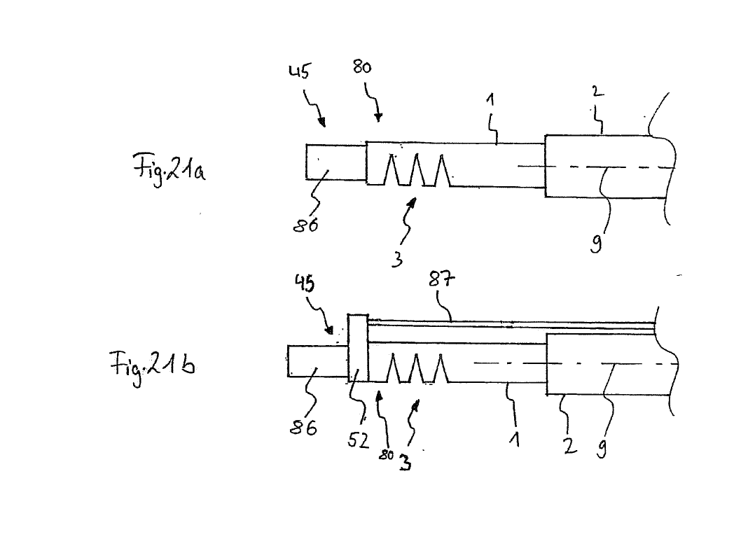

Many different types of mechanical connections 29 between the tension element 4 and the outer tube element 2 may be used. In one embodiment, as shown in FIGS. 13a and 13f, the tension element 4 may be guided in the inner tube element 1 through a lateral opening 33, wherein the tension element 4 has a distal enlargement 34, such as, for example, a knob or a component part which is mechanically fixed to the tension element 4 and which blocks a passing of the distal end 82 of the tension element 4 through the lateral opening 33 in the inner tube element 1. The distal enlargement may be located on the inner surface of the inner tube element, as shown in FIG. 13a, or on the outer surface of the inner tube element 1, as shown in FIG. 13f. In another embodiment, as shown in FIG. 13b, the distal end 82 of the tension element 4 is connected to the inner tube element 1 on the outer surface of the inner tube element 1, wherein the tension element 4 is led from the proximal end 6 of the instrument 8 to the outer surface of the inner tube element 1. In still another embodiment, as shown in FIG. 13g, the distal end 82 of the tension element 4 may be connected to the inner tube element 1 on the outer surface of the inner tube element 1, wherein the tension element 4 is led from the proximal end 6 of the instrument 8 along the inner surface of the inner tube element 1 and is led, over the front surface 15 of the inner tube element 1, to the outer surface of the inner tube element 1. In another embodiment, as shown in FIG. 13h, the distal end 82 of the tension element 4 may be connected to the inner tube element 1 on the outer surface of the inner tube element 1, wherein the tension element 4 is led from the proximal end 6 of the instrument 8 to the inner surface of the inner tube element 1 and is led, through the wall of the inner tube element 1, to the outer surface of the inner tube element 1. In another embodiment, the distal end 82 of the tension element 4 may be embedded in the wall of the inner tube element 1, as shown in FIG. 13d. In yet another embodiment, the distal end 82 of the tension element 4 may be connected to the inner tube element 1 on the inner surface of the inner tube element 1, wherein the tension element 4 is led from the proximal end 6 of the instrument 8 to the inner surface of the inner tube element 1, as shown in FIG. 13i. In another embodiment, the distal end 82 of the tension element 4 is connected to the inner tube element 1 on the inner surface of the inner tube element 1, wherein the tension element 4 is fed from the proximal end 6 of the instrument 8 along the outer surface of the inner tube element 1 and is led, over the front surface 15 of the inner tube element 1, to the inner surface of the inner tube element 1, as shown in FIG. 13c. In still another embodiment, the distal end 82 of the tension element 4 may be connected to the inner tube element 1 on the inner surface of the inner tube element 1, wherein the tension element 4 is led from the proximal end 6 of the instrument 8 to the outer surface of the inner tube element 1 and is led, through the wall of the inner tube element 1, to the inner surface of the inner tube element 1, as shown in FIG. 13e. In another embodiment, as shown in FIG. 13j, the distal end 82 of the tension element 4 may include a loop 18 configured to be guided in a first tension element guide 35 of the front surface 15 of the inner tube element, for example, around the distal opening 10 of the inner tube element 1.

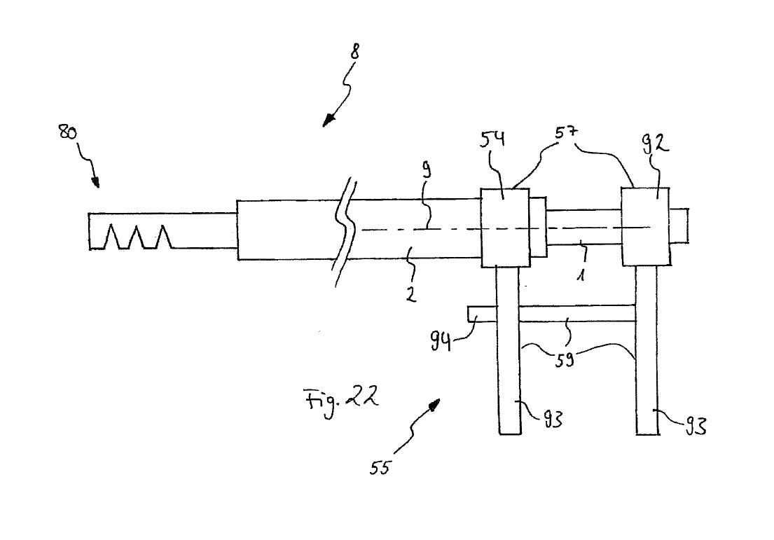

As shown in FIG. 14, the bending of the instrument tip 80 may be effected through the first structures 3 which are, upon actuation of the tension element 4, bent around one or more axes, in the preferred bending direction, for example, by local deformation of the inner tube element 1 or by hinge elements 17 (see FIG. 5). In this case, the second tension element guide 28 is designed so that a force acting upon the tension element 4 is transmitted to the first structures 3 so that a uniform bending of the instrument tip 80 is obtained. In this case, the second tension element guide 28 is designed so that the tension element 4 follows the bending occurring when the instrument tip 80 is bent. Consequently, the eyelets 28 provided between the notches on the tube segments may be wedge-like or may be designed with such thin walls that, in case the first structures are bent at a maximum bend angle, the faces of the notches abut against each other without being influenced by the eyes.

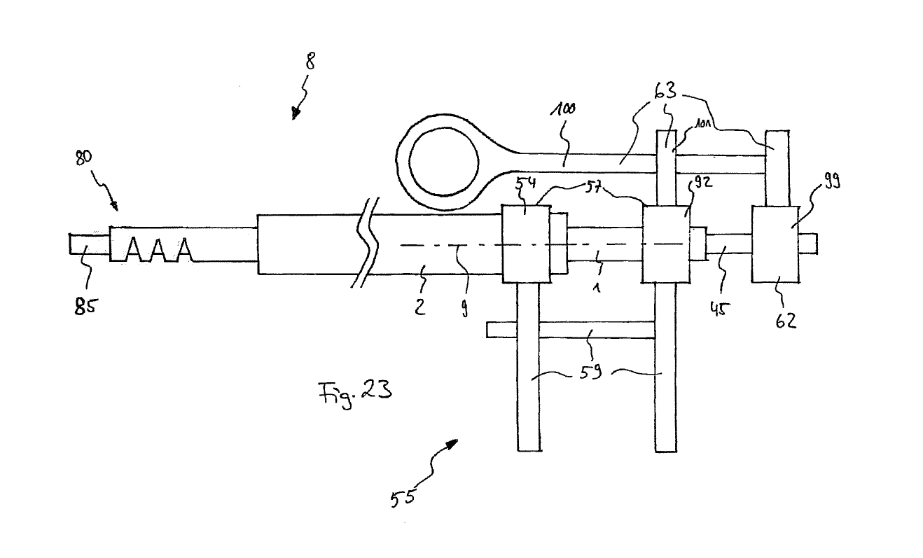

The tension element guide 28 may include many different configurations for supporting or retaining the tension element 4, including, for example, the use of one or more guide members. In one embodiment, as shown in FIG. 15a, a guide member 36 has a tubular cross-section, is disposed on the outer surface of the inner tube element 1 and guides the tension element 4 in the interior of the tubular cross-section. In another embodiment, as shown in FIG. 15d, a guide member 36 has a tubular cross-section, is disposed on the inner surface of the inner tube element 1 and guides the tension element 4 in the interior of the tubular cross-section. In another embodiment, as shown in FIG. 15b, a guide member 36 has a U-shaped cross-section, is fixed to the outer surface of the inner tube element 1 so that the open side of the U-shaped cross-section abuts against the outer surface of the inner tube element 1, and guides the tension element 4 in the interior of the U-shaped cross-section. In still another embodiment, as shown in FIG. 15e, a guide member 36 has a U-shaped cross-section, is fixed to the inner surface of the inner tube element 1 so that the open side of the U-shaped cross-section contacts the inner surface of the inner tube element 1, and guides the tension element 4 in the interior of the U-shaped cross-section. In yet another embodiment, as shown in FIG. 15c, the outer surface of the inner tube element 1 has a groove 84 and a guide member 36, wherein the tension element 4 is guided in the groove 84 and the open side of the groove 84 is, at least partially, covered by the guide member 36. In another embodiment, as shown in FIG. 16a, the wall of the inner tube element 1 has a through bore 38 which extends in parallel with the axis 9 of the instrument 8 and in which the tension element 4 is guided. In another embodiment, the inner surface of the inner tube element 1 has a groove 37 in which the tension element 4 is guided, as shown in FIG. 16b. In still another embodiment, the inner surface of the inner tube element 1 has a groove 37 in which the tension element 4 is guided and which is, at least partially, covered by a guide member 36, as shown in FIG. 16c. In another embodiment, the tension element 4 may be guided in the interior of the inner tube element 1, as shown in FIG. 16d. In yet another embodiment, the tension element 4 may be guided through a second lateral bore 40 in the wall of the inner tube element 1, which is arranged proximally with respect to the first structures 3, as shown in FIG. 17.

For transmitting the force for bending the instrument tip 80 from the proximal end 6 of the instrument 8 to the tension element 4, the inner tube element 1 and the outer tube element 2 are displaced against each other parallel to the axis 9 of the instrument 8. In order to generate an axially symmetrical cross-section, the inner tube element 1 is guided in the outer tube element 2. In this case, the outer surface 43 of the inner tube element 1 may be in direct contact with the inner surface 42 of the outer tube element 2. It may be desirable to reduce friction between the outer surface 43 of the inner tube element 1 and the inner surface 42 of the outer tube element 2 by reducing the contact surface. Further, it can be advantageous to design the cross-sectional shape of the inner tube element 1 and the cross-sectional shape of the outer tube element 2 so that rotation of the inner tube element 1 in relation to the outer tube element 2 around the axis 9 of the instrument 8 is blocked.

Many different configurations of inner and outer tube elements 1, 2 may be utilized. In one embodiment, the outer surface 43 of the inner tube element 1 and the inner surface of the outer tube element 2 have a circular cross-section, as shown in FIG. 18a. In another embodiment, the outer surface 43 of the inner tube element 1 and the inner surface of the outer tube element 2 have cross-sectional shapes which block a rotation of the inner tube element 1 in relation to the outer tube element 2 around the axis 9 of the instrument 8. These cross-sectional shapes may include any suitable configuration and, for example, may be polygonal or star-shaped. Optionally, these cross-sectional shapes may be rounded off. One example of such a configuration is illustrated in FIG. 18b.

In another embodiment, the inner surface 42 of the outer tube element 2 has a circular cross-section and the outer surface 43 of the inner tube element 1 has a cross-sectional shape which is not circular, such as, for example, a polygon, or star-shaped configuration. Optionally, this cross-sectional shape may be rounded off. One example of such a configuration is illustrated in FIG. 18c.

In still another embodiment, the outer surface 43 of the inner tube element 1 has a circular cross-section and the inner surface 42 of the outer tube element 2 has a cross-sectional shape which is not circular, such as, for example, a polygon, or star-shaped configuration. Optionally, this cross-sectional shape may be rounded off. One example of such a configuration is illustrated in FIG. 18d.

Referring now to FIG. 19, an instrument 8 may serve for guiding/inserting a second medical device 45 having a surgical effector 48. The surgical effector 48 may include, for example, grasping forceps, biopsy forceps, a needle holder, a suture appliance, a clamp applicator, scissors, a loop, a bag, a clip applicator, an injection needle, a blade, a screen device, an illumination unit, a high-frequency current cutting device, a laser cutting device, a balloon applicator, a stent applicator, a water jet dissection device, a high-frequency coagulator, an argon plasma coagulator, an ultrasonic coagulator, a camera unit, a hook device, a spraying device, a rinsing device, a suction device, an electrode, or a sensory probe.

An example of a second device 45 is a surgical instrument 85 having a surgical effector 48, a shaft 47 and a fifth operating element 46. In one such embodiment, the fifth operating element 46 is connected to the surgical effector 48 through a flexible shaft 47, as shown in FIG. 39. By actuating the fifth operating element 46, the desired function of the surgical effector 48 can be adjusted. In the illustrated embodiment of FIG. 19, the fifth operating element 46 is located at the proximal end 49 of the surgical instrument 85 and the surgical effector 48 is provided at the distal end 50 of the surgical instrument 85. The surgical instrument 85 may be positioned in the instrument 8 so that the shaft 47 of the surgical instrument 85 is guided in the inner tube element 1 and the distal end 50 of the surgical instrument 85 can be optionally led out of the instrument tip 80.

In the illustrated embodiment, the surgical instrument 85 is movable/shiftable parallel to the axis 9 of the instrument 8. The outer surface 51 of the surgical instrument 85 and the inner surface 44 of the inner tube element 1 may be in direct contact with each other. It may be desirable to reduce friction between the outer surface 51 of the surgical instrument 85 and the inner surface 44 of the inner tube element 1 by reducing the contact surface. Furthermore, it may be desirable to design the cross-sectional shape of the outer surface 51 of the surgical instrument 85 and the cross-sectional shape of the inner surface 44 of the inner tube element 1 so that a rotation of the surgical instrument 85 in relation to the inner tube element 1 around the axis 9 of the instrument 8 is blocked.

Many different configurations of inner tube elements 1 and surgical instruments 85 may be utilized. In one embodiment, the outer surface 51 of the surgical instrument 85 and the inner surface 44 of the inner tube element 1 may each have a circular cross-section, as shown in FIG. 20a. In another embodiment, the outer surface 51 of the surgical instrument 85 and the inner surface 44 of the inner tube element 1 may have cross-sectional shapes which block a rotation of the surgical instrument 85 in relation to the inner tube element 1 around the axis 9 of the instrument 8. These cross-sectional shapes may include any suitable configuration, including, for example, polygonal or star-shaped. Optionally, these cross-sectional shapes can have curvatures. One example of such a configuration is illustrated in FIG. 20b. In another embodiment, the inner surface 44 of the inner tube element 1 may have a circular cross-section and the outer surface 51 of the surgical instrument 85 may have a cross-sectional shape which is not circular, such as, for example, polygonal or star-shaped. Optionally, this cross-sectional shape may be rounded off. One example of such a configuration is illustrated in FIG. 20c. In still another embodiment, the outer surface 51 of the surgical instrument 85 may have a circular cross-section and the inner surface 44 of the inner tube element 1 may have a cross-sectional shape which is not circular, such as, for example, polygonal or star-shaped. Optionally, this cross-sectional shape may be rounded off. One example of such a configuration is illustrated in FIG. 20d.

A further example of a second device 45, shown, for example, in FIGS. 21a and 21b, relates to a surgical effector 86 which is fixed to the instrument tip 80. The surgical effector 86 may include, for example, grasping forceps, biopsy forceps, a needle holder, a suture appliance, a clamp applicator, scissors, a loop, a bag, a clip applicator, an injection needle, a blade, a screen device, an illumination unit, a high-frequency current cutting device, a laser cutting device, a balloon applicator, a stent applicator, a water jet dissection device, a high-frequency coagulator, an argon plasma coagulator, an ultrasonic coagulator, a camera unit, a hook device, a spraying device, a rinsing device, a suction device, an electrode or a sensory probe. Upon bending of the instrument tip 80, the surgical effector 86 may follow the bending. In this way, the surgical effector 86 may be aligned/orientated.

In one embodiment, as shown in FIG. 21b, the surgical effector 86 is connected to the instrument tip 80 through a fourth device 52. The fourth device 52 allows rotation of the surgical effector 86 around the axis 9 of the instrument 8.

An example of the design of the fourth device 52 has mechanical gear elements 39 (shown for example, in FIG. 31a) which are designed so that the rotation of the surgical effector 86 can be adjusted via a load transmission element 87 in the form of a bending-flexible rotating shaft, for example. In this case, the load transmission element 87 leads from the fourth device 52 to the proximal end 6 of the instrument 8. The fourth device 52 is designed so that the rotation of the surgical effector 86 can be adjusted by application of force or torque to the load transmission element 87.

In another embodiment including the fourth device 52, shown in FIG. 38b, the force transmitting member 87 has a sixth operating element 112 which is connected to the force transmitting member 87 so that, upon actuation of the sixth operating element 112, the rotation of the surgical effector 86 can be adjusted.

In another embodiment, shown in FIG. 31a, mechanical gear elements 39 include two spur gears where a first spur gear 88 is supported so as to be rotatable around the axis 9 of the instrument 8 and connected to the surgical effector 86 so as to be integrally rotatable therewith, and where a second spur gear 89 is rotatably supported so as to form a gear unit with the first spur gear 88, and which is connected to the force transmitting member 87 so as to be integrally rotatable therewith. In this case, the force transmitting member 87 is designed as a shaft so that it transmits a torque applied to the force transmitting member 87 at the proximal end 6 of the instrument 8 to the second spur gear 89. The first spur gear 88 may have a larger diameter than the second spur gear 89 in order to set a gear transmission ratio greater than one between the rotational speeds of the force transmitting member 87 and the surgical effector 86.

In another embodiment, as shown in FIG. 31b, mechanical gear elements 39 include two bevel gears, a first bevel gear 90 of which is supported so as to be rotatable around the axis 9 of the instrument 8 and connected to the surgical effector 86 so as to be integrally rotatable therewith, and a second bevel gear 91 pivotally supported so as to form a gear unit with the first bevel gear 90, and operatively connected to the force transmitting member 87. In this case, the force transmitting member 87 may be designed as a belt drive so that it transmits a tensile force applied to the force transmitting member 87 at the proximal end 6 of the instrument 8 to the second bevel gear 91 such that the second bevel gear 91 is rotated. The force transmitting member 87 may be designed as a tackle line, wherein the rotational axis of the second bevel gear 91 is substantially vertical to the instrument axis. The first bevel gear 90 may have a larger diameter than the second bevel gear 91 in order to set a transmission ratio greater than one between the rotational speeds of the second bevel gear 91 and the first bevel gear 90 and, thus, to reduce the tensile force in the force transmitting member 87, which may provide for a desired torque of the surgical effector 86.

Optionally, the surgical effector 86 may be provided with a first control element 53 and a seventh operating element 111, as shown in FIG. 38a. The seventh operating element 111 is connected to the first control element 53 so that, upon actuation of the seventh operating element 111, the desired function of the surgical effector 86 can be adjusted preferably from the proximal end 6 of the instrument 8.

In one embodiment, the first control element 53 can be optionally set so that the adjustment of the desired function of the surgical effector 86 is blocked. Thus, a preferred adjustment of the desired function of the surgical effector 86 can be maintained without actuation of the seventh operating element 111. In another embodiment, the first control element 53 may include a metal wire by which, optionally, a linear force or a torque can be transmitted from the seventh operating element 111 to the surgical effector 86. In still another embodiment, the first control element 53 may include a thread through which a tensile force can be transmitted from the seventh operating element 111 to the surgical effector 86. In another embodiment, the first control element 53 may include at least one electrically conductive cable through which the electrical signals can be transmitted from the seventh operating element 111 to the surgical effector 86. These electrical signals may, for example, include analogous measurement signals such as voltages or currents, digital data, or high-frequency current for operating a high-frequency effector.

In one embodiment, the fourth device 52 may be configured such that the adjustment of the rotation of the surgical effector 86 may alternatively be blocked. In this way, a preferred alignment/orientation of the fourth device 52 without actuation of the sixth operating element 112 may be maintained.

Control Device.

A control device 55, shown, for example, in FIG. 22, may serve for the manual control of the instrument 8 in various designs. As contemplated by the present application, the control device 55 may be adapted for many different functions, including, for example, one or more of the following functions: the manual adjustment of the bending of the instrument tip 80, the manual adjustment of the advancing of a surgical instrument 85 parallel to the axis 9 of the instrument 8, the manual adjustment of the rotation of a surgical instrument 85 around the axis 9 of the instrument 8, the manual adjustment of the desired function of a surgical effector 48, manual adjustment of the rotation of a surgical effector 86, the manual adjustment of the desired function of a surgical effector 86, the manual adjustment of the advancing of the instrument 8 parallel to the axis 9 of the instrument 8, and the manual adjustment of the rotation of the instrument 8 around the axis 9 of the instrument 8.

The illustrated control device 55 may include a seventh device 57 for applying a thrust force to the inner and outer tube elements of the instrument and a first operating element 59 for the manual/electromotive application of a thrust force. The seventh device 57 may include two connecting elements, which may be in the form of clamps, connector rings, or other such components, wherein the first connecting element 54 establishes a connection between the first operating element 59 and the outer tube element 2 of the instrument 8, and the second connecting element 92 establishes a connection between the first operating element 59 and the inner tube element 1 of the instrument. The first connecting element 54 and the second connecting element 92 may be designed so that they are movable in relation to each other in a direction parallel to the axis 9 of the instrument 8. In this case, the first connecting element 54 and the second connecting element 92 as well as the first operating element 59 are coupled to each other so that, by actuation of the first operating element 59, a movement of the first connecting element 54 in relation to the second connecting element 92 can be adjusted. The first connecting element 54 and the second connecting element 92 may be connected to the instrument 8 so that, upon movement of the first connecting element 54 in relation to the second connecting element 92, the inner tube element 1 is displaced in relation to the outer tube element 2, whereby a bending of the instrument tip 80 can be adjusted.

In the embodiment of FIG. 22, the first operating element 59 includes two rod-shaped or trigger-shaped handles or grips 93, where one is integrally connected to the first connecting element 54 and the other is integrally connected to the second connecting element 92. By moving the grips 93 in relation to each other, the first connecting element 54 can be displaced in relation to the second connecting element 92. In this way, the bending of the instrument tip 80 can be adjusted. Optionally, the movement of the grips 93 can be guided by a guiding element 94. The guiding element may include, for example, a guide rod which extends along the moving direction of both grips and which is fixedly attached to one grip and slidably supported in/on the other grip.

In another embodiment, as shown in FIG. 32, the first operating element 59 has two rod-shaped handles/grips 93, one of which is integrally connected to the first connecting element 54 and the other is fixedly connected to the second connecting element 92. In addition, the two grips 93 are pivotally connected to each other through a pin or bolt 95 according to the scissors principle. The first connecting element 54 can be displaced in relation to the second connecting element 92 by turning both grips 93 in relation to each other around the pin 95. In this way, the bending of the instrument tip 80 can be adjusted.

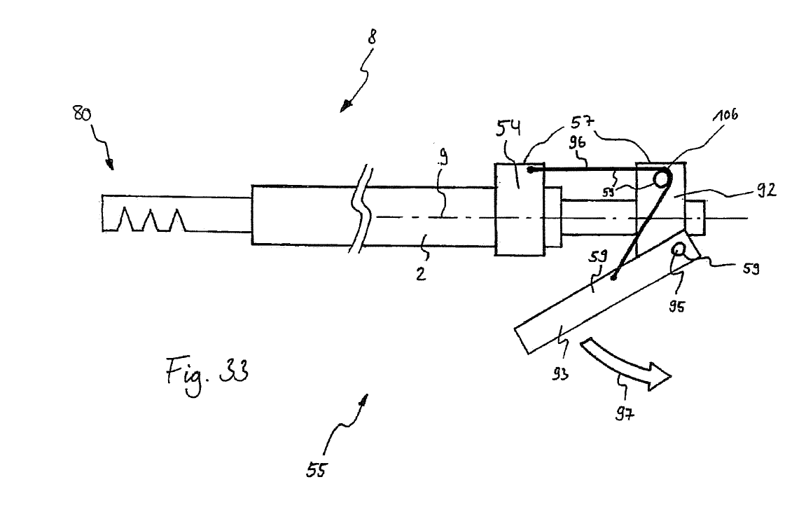

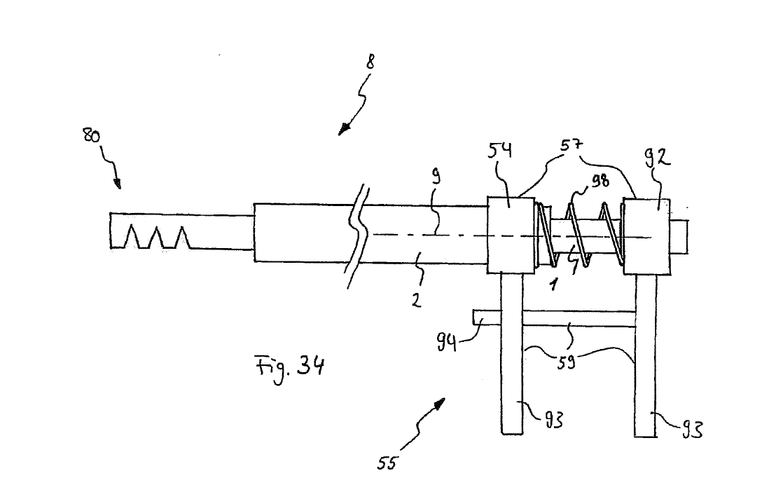

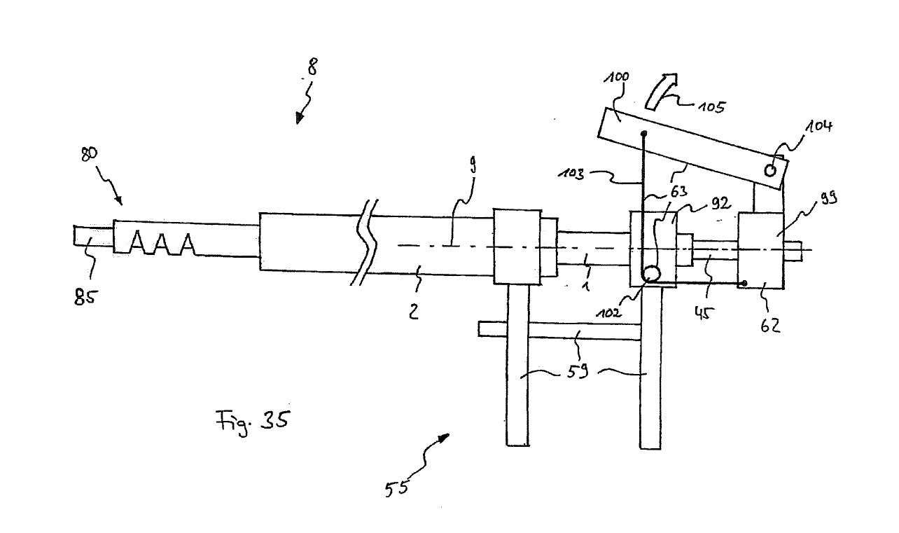

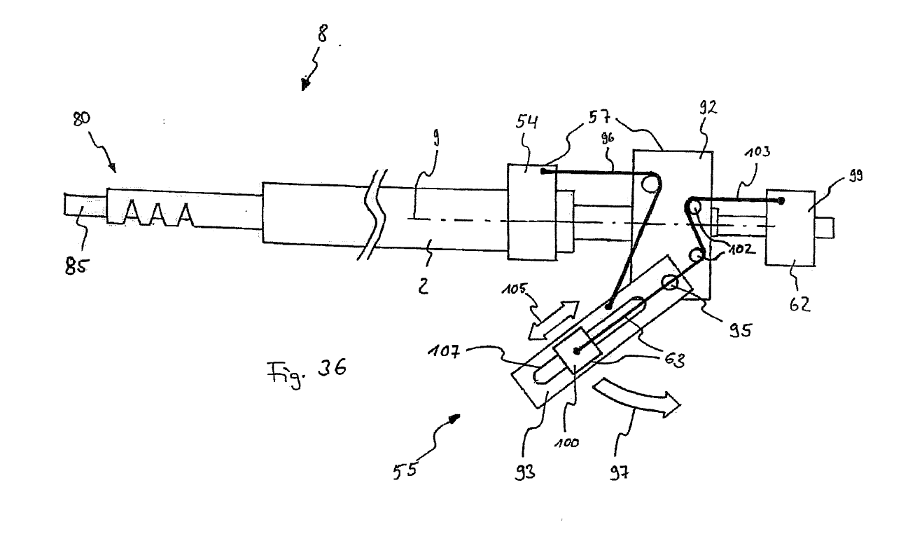

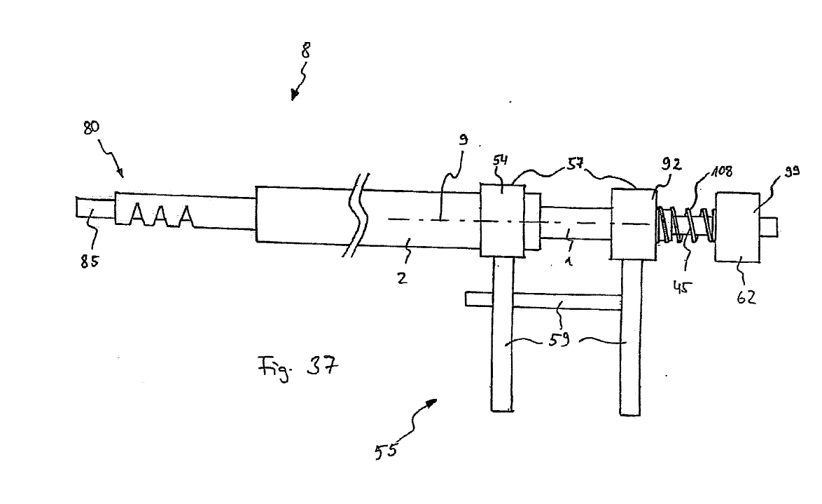

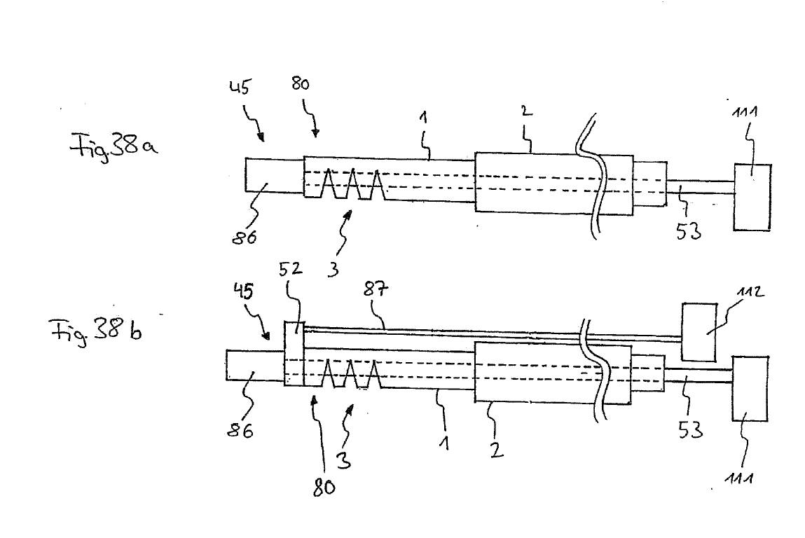

In another embodiment, as shown in FIG. 33, the first operating element 59 includes a handle/grip 93 in the form of a trigger which is on one side, through a pivot 95, pivotally connected to a connecting element 57. Furthermore, the grip 93 may be operatively connected to the other connecting element through a second force transmitting member 96 in the form of a tackle line so that, when turning the grip 3 around the pivot 95 in a pivot direction 97, the first connecting element 54 is moved towards the second connecting element 92. For doing so, the tackle line 96 is fixed to a center portion of the trigger or lever 59 and guided over a deflection device 106 which is disposed on the connecting element carrying the pivot 95. In another embodiment, the first operating element includes gear elements, such as, for example, a gear wheel, a gear rod or a gear belt. In still another embodiment, the first operating element may include at least one lever mechanism.

In an example of an embodiment of the deflection device 106, shown, for example, in FIG. 33, the deflection device 106 includes a deflection roll pivotally attached to the one connecting element, in which the force transmitting member 96 is guided. In a further example of an embodiment of the deflection device 106, the deflection device 106 may include a static mechanical barrier deforming the force transmitting member 96. In a further example of an embodiment of the deflection device 106, the deflection device 106 includes a tube element on which the force transmitting member 96 is guided.

In an example of an embodiment of the force transmitting member 96, as shown in FIG. 33, the force transmitting member 96 includes a pull thread or a pull cable. In a further example of an embodiment of the force transmitting member 96, the force transmitting member 96 includes a wire.

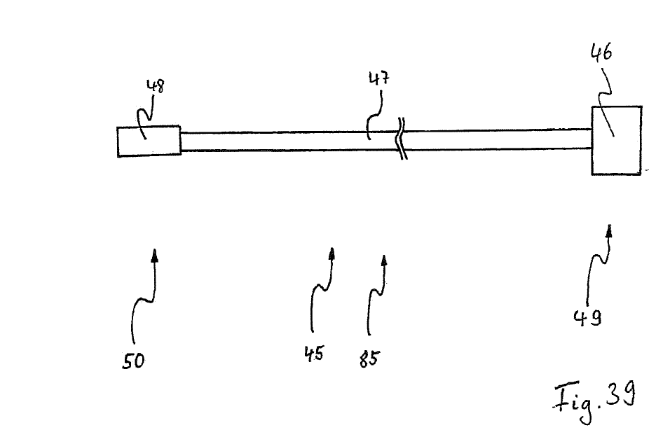

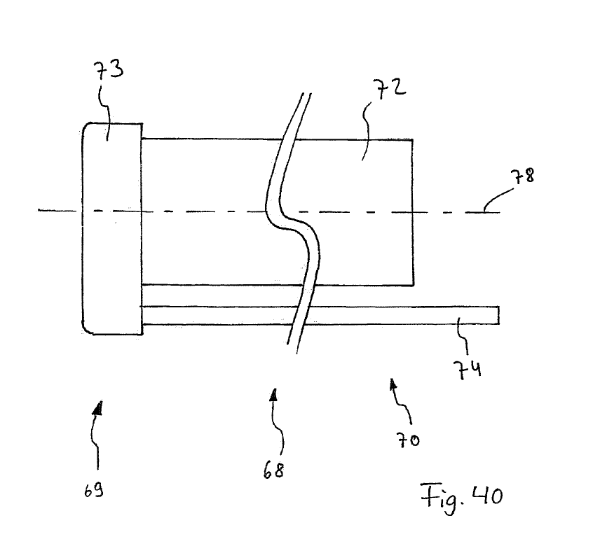

In one embodiment, a seventh device 57 has a spring element 98 which is arranged between the two connecting elements and which is compressed when the first connecting element 54 approaches the second connecting element 92, as shown in FIG. 34.

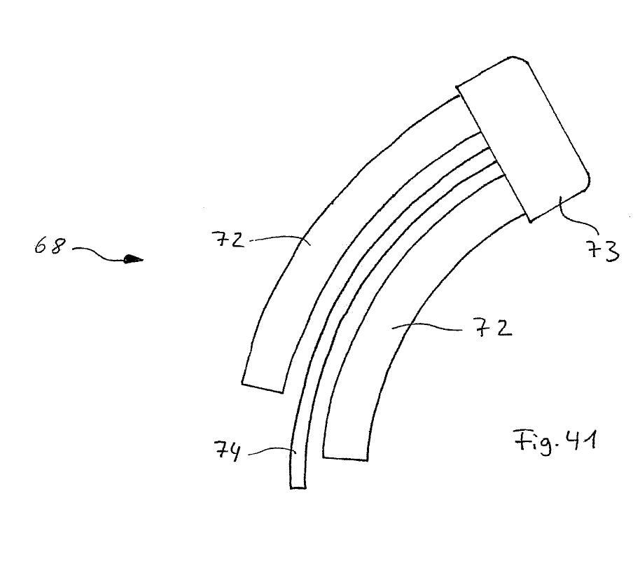

As shown in FIG. 35, the control device 55 may optionally include an eighth advancing device 62 and a second operating element 63. The illustrated eighth advancing device 62 has a third connecting element 99 which may be a clamping ring-like connecting element 99, and which is connected to the surgical instrument 85. The connection between the third connecting element 99 and the surgical instrument 85 may be designed so that a movement of the third connecting element 99 along the axis 9 of the instrument 8 effects a movement of the surgical instrument 85 along the axis 9 of the instrument 8. In this case, the operating element 63 serves for a longitudinal displacement of the third connecting element 99 and, thus, for a longitudinal displacement of the surgical instrument 8.

In one embodiment, as shown in FIG. 23, the second operating element 63 has a pull rod or a tackle line, each having a grip 100 which is hinged to the third connecting element 99 through a pin so that, by moving the pull rod 100, the third connecting element 99 can be displaced parallel to the axis 9 of the instrument 8. Thus, the surgical instrument 85 can be moved in relation to the instrument 8. Optionally, the pull rod having a grip 100 can be guided by a guide element 101, for example, in the form of an eye or a sleeve which is preferably attached to the connecting element of the inner tube element of the instrument.

In another embodiment, as shown in FIG. 35, the second operating element 63 has a lever-shaped grip 100 which is pivotally connected to the third connecting element 99 through a turning device 104. A force transmitting member 103 preferably in the form of a pull cable is fixed to a center portion of the grip 100. The force transmitting member 103 is further connected to the third connecting element 99 so that, upon pivoting of the grip 100 in a desired direction 105, the third connecting element 99 can be moved in relation to the connecting element of the inner tube element parallel to the axis 9 of the instrument 8. The force transmitting member 103 can be optionally deflected via one or more deflection devices 102 which are arranged on the connecting element of the inner tube element.

In still another embodiment, the second operating element has gear elements such as a gear wheel, a gear rod or a gear belt. In another embodiment, the second operating element has at least one lever mechanism.

In an example of an embodiment of the deflection device 102, the deflection device 102 consists of a deflection roll pivotally supported at the connecting element of the inner tube element, on which the pull cable-like force transmitting member 103 is guided. In a further advantageous embodiment of the deflection device 102, the deflection device 102 consists of a static mechanical barrier deforming the force transmitting member 103. In a further example of an embodiment of the deflection device 102, the deflection device 102 consists of a tube element on which the force transmitting member 103 is guided.

In an example of an embodiment of the force transmitting member 103, the force transmitting member 103 is a pull thread or a pull cable. In a further example of an embodiment of the force transmitting member 103, the force transmitting member 103 is a wire.

In the illustrated embodiment of FIG. 36, the first operating element 59 has a lever-shaped grip 93 which is hinged to the second connecting element 92 of the inner tube element of the instrument. Furthermore, the grip 93 is operatively connected to the first connecting element 54 of the outer tube element of the instrument through the second pull cable-like force transmitting member 96 and the deflection device arranged on the first connecting element 54 so that, when pivoting the grip 63 around the hinge 95 in the preferred direction of rotation 97, the first connecting element 54 is pulled to the second connecting element 92. The force transmitting member 96 can be optionally deflected, as already indicated, by one or several deflection devices 106. According to this embodiment, the second operating element may be integrated into the first operating element 59. For doing so, the first operating element further has a guiding means along the lever-shaped first operating element which is designed so that it can accommodate the grip 100 of the second operating element 63. The guiding means 107, here in the form of a longitudinal slot, allows a movement of the grip 100 of the second operating element 63, for example, along the grip 93 of the first operating element 59. A cable-like force transmitting member 103 may be connected to an area of the grip 100 of the second operating element. Furthermore, the force transmitting member 103 may be connected to the third connecting element 99 of the surgical instrument so that, upon moving of the grip 100 of the second operating element 63 in a desired direction 105, the third connecting element 99 can be displaced parallel to the axis 9 of the instrument 8 in relation to the second connecting element 92. For doing so, the force transmitting member 103 may be deflected via one or several deflection devices 102 which are arranged on the second connecting element 92.

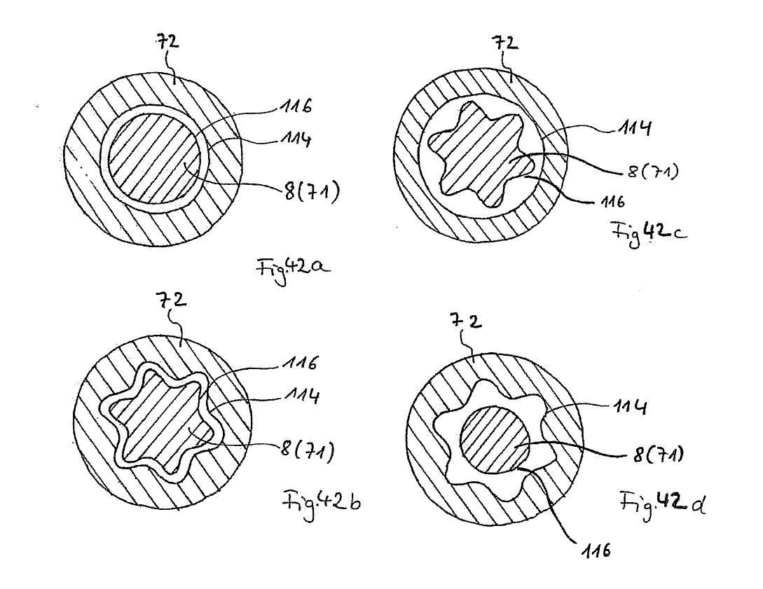

In one embodiment, as shown in FIG. 37, the eighth device 62 has a spring element 108 which is arranged between the second and third connecting element and by which, upon moving the third connecting element 99 in relation to the second connecting element 92, a reset force can be exerted on the third connecting element 99.

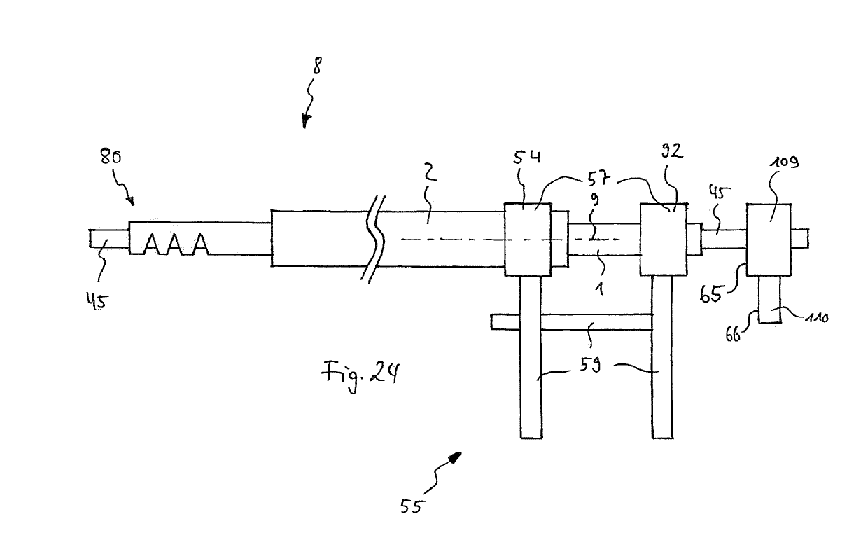

As shown in FIG. 24, the control device 55 may include a ninth device 65 and a third operating element 66 for actuating the surgical instrument. For this purpose, the ninth device 65 may include a fourth connecting element, preferably in the form of a clamping ring 109, which is connected to the surgical instrument 85. In this case, the mechanical connection between the fourth connecting element 109 and the surgical instrument 85 is designed so that a rotation of the fourth connecting element 109 around the axis 9 of the instrument 8 effects a rotation of the surgical instrument 85 around the axis 9 of the instrument 8. Furthermore, the third operating element 66 may be operatively connected to the fourth connecting element 109 so that an actuation of the third operating element 66 can effect a rotation of the fourth connecting element 109 around the axis 9 of the instrument 8.

In one embodiment, the third operating element 66 has a rod-shaped grip 10 which is operatively connected to the fourth connecting element 99 so that, by rotation of the grip 10, the fourth connecting element 109 can be turned around the axis 9 of the instrument 8. In this way, the surgical instrument 85 can be rotated around the axis 9 of the instrument 8. In this case, it is not mandatory that the axis of the grip 10 corresponds to the axis 9 of the instrument 8 but can, by arranging a deflection gear unit therebetween, also be aligned at an angle with respect to the longitudinal axis of the instrument 8. In another embodiment, for this purpose, the third operating element 66 has gear elements, e.g. a gear wheel, a gear rod or a gear belt. In still another embodiment, the third operating element 66 has at least one lever mechanism. In this case the eighth device 62 and the ninth device 65 may be combined so that control of the rotation of the surgical instrument 85 around the axis 9 of the instrument 8 and control of the movement of the surgical instrument 85 parallel to the axis 9 of the instrument 8 is effected through one single connecting element which can be connected to the surgical instrument 85 so that a rotation around the axis 9 of the instrument 8 as well as a movement parallel to the axis 9 of the instrument 8 can be transmitted to the surgical instrument 85.

In another embodiment, the seventh device 57 may optionally be decoupled from the first operating element 59. In this way, a working point adjustment of the first operating element 59 is possible. In another embodiment, the seventh device 57 may be configured such that the adjustment of the distance between the first connecting element 54 and the second connecting element 92 can be optionally blocked. In this way, a preferred bending of the instrument tip 80 can be maintained without actuation of the first operating element 59.

In another embodiment, the eighth device 62 can be optionally decoupled from the second operating element 63. By this, a working point adjustment of the second operating element 63 is possible. In still another embodiment, the adjustment of the movement of the eighth device 62 parallel to the axis 9 of the instrument 8 can be optionally blocked. By this, a preferred position of the surgical instrument 85 can be maintained without actuation of the second operating element 63.

In one embodiment, the ninth device 65 can be optionally decoupled from the third operating element 66. By this, a working point adjustment of the third operating element 66 is possible. In another embodiment, the adjustment of the rotation of the ninth device 65 around the axis 9 of the instrument 8 can be optionally blocked. By this, a preferred position of the surgical instrument 85 can be maintained without actuation of the third operating element 66.

In another embodiment, an endoscopic system uses a surgical instrument 85 as second device 45, and the fifth operating element 46 is connected to the control device 55. This design allows an adjustment of the desired function of the surgical instrument 85 through the fifth operating element 46 together with an operation of the instrument 8 through the first operating element 59, optionally through the second operating element 63 and optionally through the third operating element 66 in the same reference system. With a suitable design of the control device 55, this allows a manual operation of the instrument 8 and an adjustment of the desired function of the surgical instrument 85, for example, in a single-handed manner.

In another embodiment, an endoscopic system uses a surgical effector 86 as second device 45, and the seventh operating element 111 is connected to the control device 55. This design allows an adjustment of the desired function of the surgical effector 86 through the seventh operating element 111 together with an operation of the instrument 8 through at least the first operating element 59 in the same reference system. With a suitable design of the control device 55, this allows a manual operation of the instrument 8 and an adjustment of the desired function of the surgical effector 86, for example, in a single-handed manner.

In still another embodiment, an endoscopic system uses a surgical effector 86 in combination with a fourth device 52 as second device 45, and the sixth operating element 112 is connected to the control device 55. This design allows an adjustment of the rotation of the surgical effector 86 through the sixth operating element 112 together with an operation of the instrument 8 through at least the first operating element 59 in the same reference system. With a suitable design of the control device 55, this allows a manual operation of the instrument 8 and an adjustment of the rotation of the surgical effector 86, for example, in a single-handed manner.

Overtube Device.

An overtube device 68, shown, for example, in FIG. 25, may serve to accommodate and insert at least one eleventh device 71, preferably an instrument 8, into the human body. In one embodiment, an overtube device 68 serves to place at least one eleventh device and a camera system or visual device in a tube-like hollow organ such as the digestive tract. As one example, two instruments 8 can be placed in the overtube device 68. The camera system can optionally consist of a flexible endoscope or of a camera unit 79 (see FIG. 29) integrated in the overtube device. The overtube device 68 has, at its distal end 69, distal openings 76 from which the inserted instruments 8 (see FIG. 26) and, optionally, the inserted flexible endoscope (not shown) can emerge. In this case, a distal end element 73 of the overtube device 68 represents the reference system of the instruments 8 and the camera system.

For inserting the overtube device 68 into a tube-like hollow organ such as the digestive tract, high flexibility of the overtube device 68 may be desired, in particular when passing through strongly curved tubular hollow organs such as the large intestine. At the same time, good control of the reference system represented by the distal end element 73 of the overtube device 68, that is control regarding alignment/orientation and positioning of the distal end element 73 of the overtube device 68, may also be desired. The combination of high flexibility of the overtube device and a good control of the distal end element 73 of the overtube device 68 from the proximal end 70 of the overtube device 68 has been difficult to achieve.

For addressing this challenge, the overtube device 68 of the present application may include a second shaft-like or cable-like control element 74 which is connected to the distal end element 73 of the overtube device 68 and which extends as far as the proximal end 70 of the overtube device 68. Thus, the second control element 74 represents a second mechanical means of influence on the distal end element 73 of the overtube device 68 from the proximal end 70 of the overtube device 68. For increasing flexibility of the overtube device 68, tube elements 72 of the overtube device 68, in which the eleventh device 71 such as an instrument 8 can be guided, may optionally be decoupled from the second control element 74 so that a local displacement of a tube element 72 in relation to the second control element 74 parallel to the axis 78 of the overtube device 68 is possible. Thus, a bending of the depicted overtube device 68 does not cause a compression of the tube elements 72 disposed in the direction of the bending and a stretching of tube elements 72 disposed in the opposite direction of the bending, by which reaction forces acting against the bending may occur, but instead may cause a local displacement of the tube elements 72 in relation to the second control element 74, as shown in FIG. 41.

The overtube device 68, as shown in FIG. 40, may have, at its distal end 69, the distal end element 73 in the form of an end cover which is connected to the second control element 74. The second control element 74 may be designed as a shaft or a cable and extends as far as the proximal end 70 of the overtube device 68. Furthermore, the overtube device has at least one tube element 72 connected to the distal end element 73.

As shown in FIG. 25, the insertion of an instrument 8 into the tube element 72 represents an example of an application of the overtube device 68. In this case, the instrument 8 represents the eleventh device 71. The instrument tip 80 of the instrument 8 preferably protrudes from the distal end 69 of the overtube device 68.

The eleventh device 71 may be movable parallel to the axis 78 (see FIG. 40) of the overtube device. The outer surface 116 of the eleventh device 71 and the inner surface 114 of the tube element 72 may be in direct contact with each other, or in other configurations, shown, for example, in FIGS. 42a-42d. It may be desirable to reduce friction between the outer surface 116 of the eleventh device 71 and the inner surface 114 of the tube element 72 by reducing the contact surface. Furthermore, it may be desirable to design the cross-sectional shape of the outer surface 116 of the eleventh device 71 and the cross-sectional shape of the inner surface 114 of the tube element 72 so that a rotation of the eleventh device 71 in the tube element 72 is blocked.

In one embodiment, as shown in FIG. 42a, the outer surface 116 of the eleventh device 71 and the inner surface 114 of the tube element 72 have a circular cross-section. In another embodiment, the outer surface 116 of the eleventh device 71 and the inner surface 114 of the tube element 72 may have cross-sectional shapes that block a rotation of the eleventh device 71 in the tube element 72. These cross-sectional shapes can include any suitable configuration and may, for example, be polygonal or star-shaped. Optionally, these cross-sectional shapes may be rounded off. One example of such a configuration is illustrated in FIG. 42b. In still another embodiment, the inner surface 114 of the tube element 72 may have a circular cross-section and the outer surface 116 of the eleventh device 71 may have a cross-sectional shape which is not circular, and may be, for example, polygonal or star-shaped. Optionally, this cross-sectional shape may be rounded off. One example of such a configuration is illustrated in FIG. 42c. In yet another embodiment, the outer surface 116 of the eleventh device 71 has a circular cross-section and the inner surface 114 of the tube element 72 has a cross-sectional shape which is not circular, and may be, for example, polygonal or star-shaped. Optionally, this cross-sectional shape may be rounded off. One example of such a configuration is illustrated in FIG. 42d.

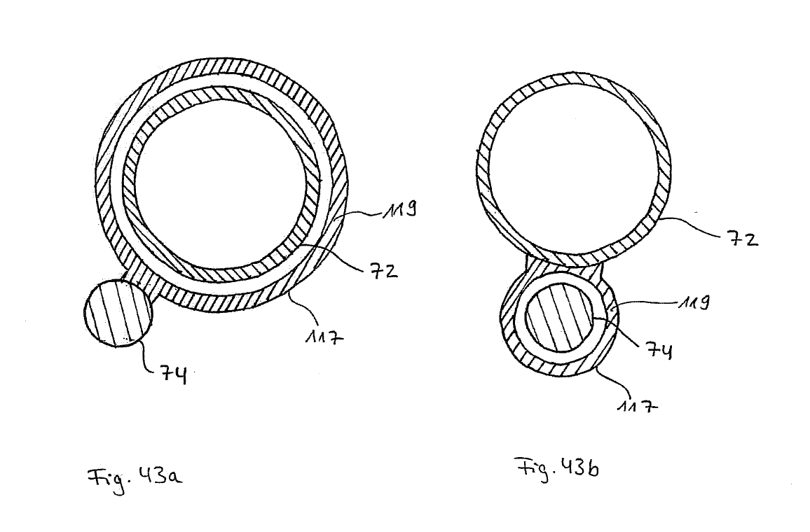

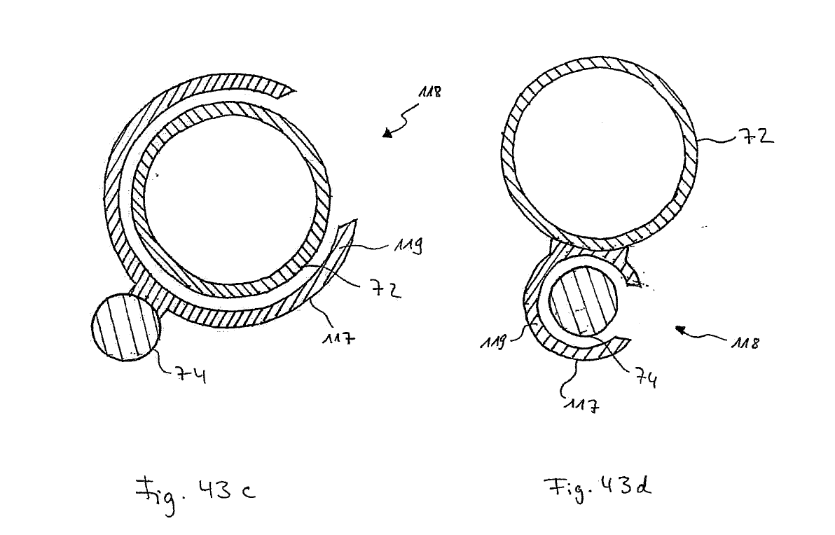

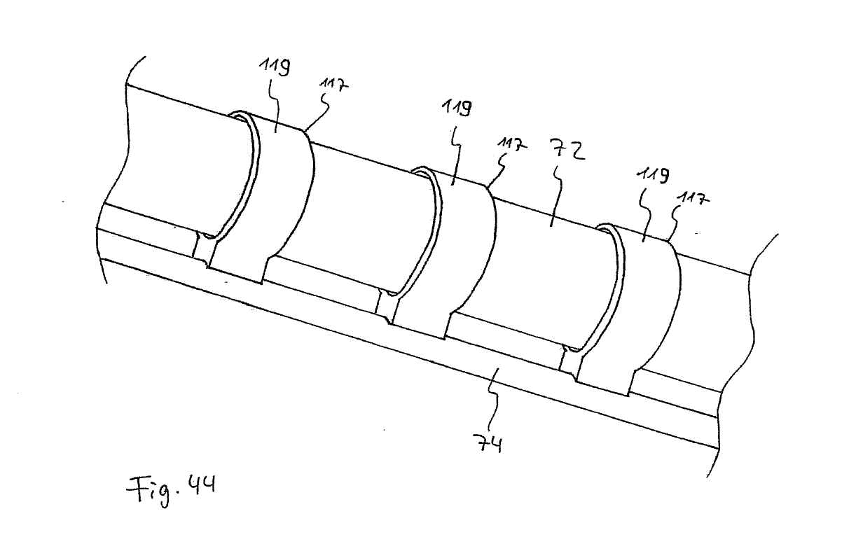

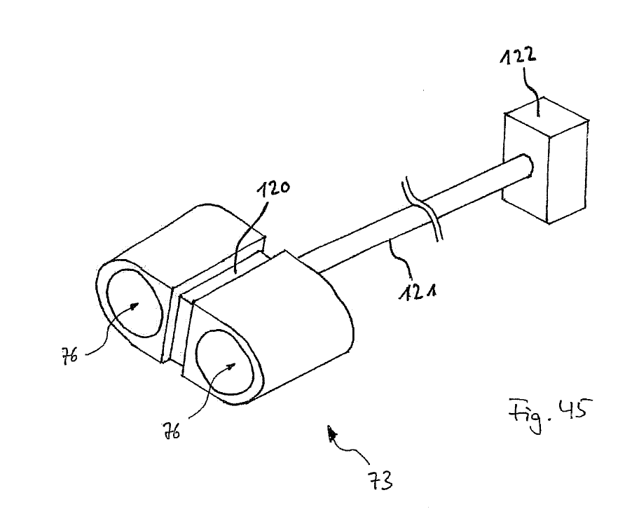

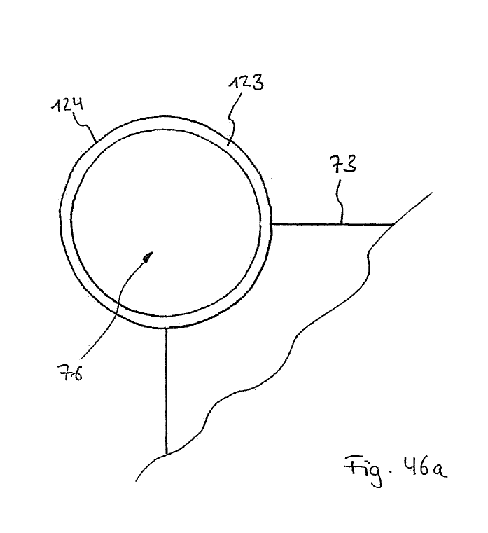

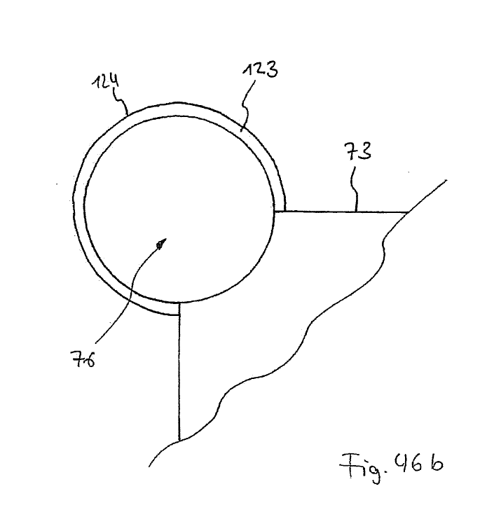

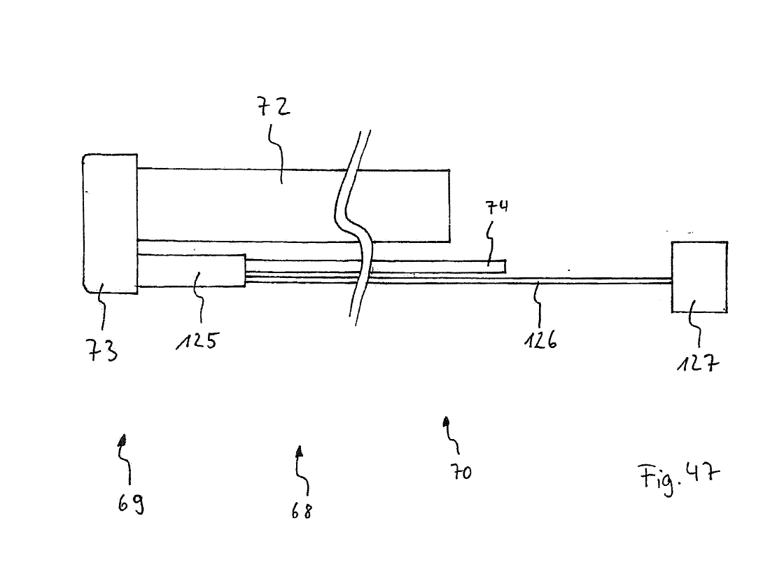

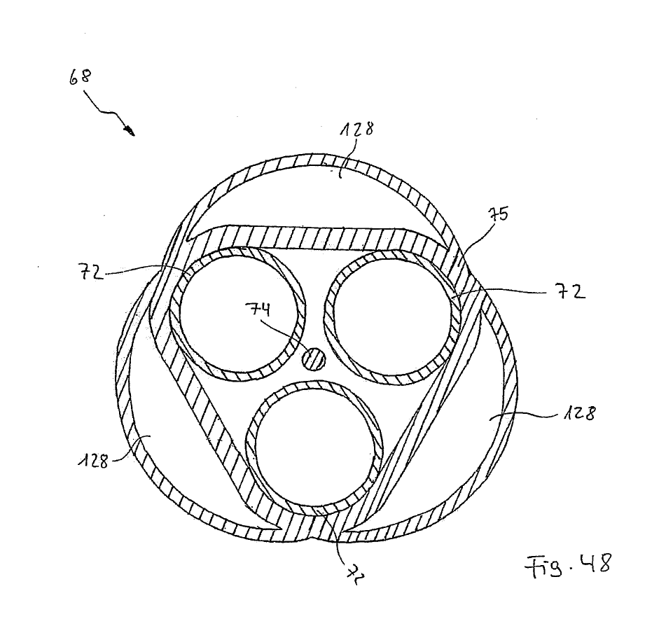

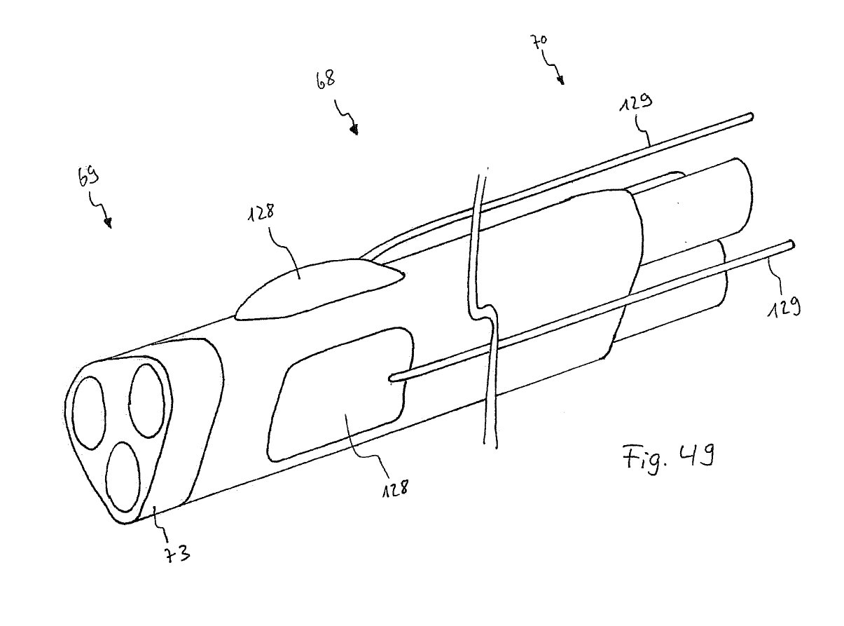

In one embodiment, an overtube device 68 includes a guiding device 117, shown, for example, in FIGS. 43a-43d. As one example, this guiding device 117 may be sleeve-shaped, extending along the overtube device and being fixedly connected to the at least one tube element 72 over the entire length of the tube. Within the sleeve-shaped guiding device, the second control element 74 may supported so as to be axially movable so that the tube element 72 can be locally displaced in relation to the second control element 74 parallel to the axis of the overtube device 68. In one such embodiment, the guiding device 117 may be designed so that the distance between the second control device 74 and the tube element 72 cannot change.

In one embodiment, the guiding device 117 includes at least one tubular or sleeve-shaped guiding segment 119. It is not mandatory that the guiding segment 119 extends continuously over the entire length of the overtube device. For example, several guiding segments or sleeves 119 can be arranged along the overtube device at regular distances (see, for example, FIG. 44), each of which is fixedly connected to the respective tube element. In another embodiment, as shown in FIG. 43a, a guiding segment 119 is fixedly connected to the second control element 74 and has a closed ring structure in which the tube element 72 is movably guided. In still another embodiment, a guiding segment 119 is fixedly connected to the tube element 72 and has a closed ring structure in which the second control element 74 is movably guided, as shown in FIG. 43b. In yet another embodiment, a guiding segment 119 is fixedly connected to the second control element 74 and has a ring structure which has a lateral opening 118 and in which the tube element 72 is movably guided, as shown in FIG. 43c. In another embodiment, a guiding segment 119 is fixedly connected to the tube element 72 and has a ring structure which has a lateral opening 118 and in which the second control element 74 is movably guided, as shown in FIG. 43d.

In an example of the design of the overtube device 68, as illustrated in FIG. 26, the overtube device 68 has three tube elements 72. The distal cover-shaped end element 73 has three distal openings 76 each of which forms a connection to one of the tube elements 72. The tube elements 72 are connected to the distal end element 73 and are held together by said element. Two of the tube elements 72 may be provided for accommodating instruments, and a further tube element 72 may be provided for accommodating a flexible endoscope (not shown).

In another example of the design of the overtube device 68, as shown in FIG. 29, the overtube device 68 may have two tube elements 72. The distal end element 73 has two distal openings 76 establishing a connection to one of the tube elements 72, respectively. The tube elements are connected to the distal end element 73 and are held together by said element. The two tube elements 72 may be provided for accommodating instruments. The distal end element 73 may include a camera unit 79 integrated therein.

In one embodiment, the camera unit 79 has a mechanical device or driving means by which the viewing angle of the camera unit 79 can be adjusted (not shown).

In another embodiment, as shown in FIG. 27, the overtube device 68 has an outer covering 75, in which the tube elements 72 extending in parallel are arranged and bundled.

In another embodiment, as shown in FIG. 28, the second cable-like control element 74 may, at its proximal end, be connected to a fourth operating element 77. In this case, the cable-like control element 74 has a predetermined torsional and bending resistance like a Bowden cable means. The connection between the second control element 74 and the fourth operating element 77 may be designed so that a rotation of the fourth operating element 77 effects a rotation of the second control element 74 and a movement of the fourth operating element 77 effects a movement of the second control element 74. By actuation of the fourth operating element 77, the orientation and position of the distal end element 73 may be controlled.

In another embodiment, as shown in FIG. 45, an end element may include at least one distal opening 76 as well as an actuating device 120 by which the position and/or the orientation of the distal opening 76 can be adjusted. The illustrated actuating device 120 has a third control element 121 and an eighth operating element 122, wherein the third control element 121 is connected to the eighth operating element 122.

In another embodiment, the actuating device 120 has a pneumatic actuator which is adapted, when compressed air is supplied, to adjust the position or orientation or the position as well as orientation of a distal opening 76. Compressed air is supplied and applied via the third control element 121. The supply and application of compressed air is controlled via the eighth operating element 122.

In another embodiment (not shown), the actuating device 120 has a hydraulic actuator which is adapted, when a liquid medium is fed or sucked off, to adjust the position or orientation or the position as well as the orientation of a distal opening 76. The liquid medium may be fed and/or sucked off via the third control element 121. The feeding and/or sucking off of a liquid medium is controlled via the eighth operating element 122.

In another embodiment (not shown), the actuating device 120 has a mechanical transmission which is adapted, upon coupling of a force and/or torque, to adjust the position or orientation or the position as well as orientation of a distal opening 76. The coupling of a force and/or torque is effected via the third control element 121. The coupling of a force and/or torque is controlled via the eighth operating element 122.

In another embodiment (not shown), the tube element 72 has a mechanism which is adapted to block the movement of an eleventh device 71 provided in the tube element 72, preferably an instrument 8 or a flexible endoscope 113. By this, a preferred position of the eleventh device 71 in the tube element 72 can be maintained by the tube element 72.

In another embodiment (not shown), the tube element 72 has a mechanism which is adapted to block a rotation of an eleventh device 71, preferably an instrument 8 or a flexible endoscope 113, which is provided in the tube element 72. By this, a preferred orientation of the eleventh device 71 in the tube element 72 can be maintained by the tube element 72.

In an example of the design of the distal opening 76 of the distal end element 73, the distal opening 76 may include a hose element 123, as shown in FIGS. 46a and 46b. The hose element 123 is attached to the distal end element 73 so that an eleventh device inserted into a tube element can emerge from the distal opening 76 at the distal end of the overtube device 68. By using a hose element made of a flexible material, such as, for example, a plastic film, the total cross-section of the distal end of the overtube device 68 can be reduced as the lumen of the distal opening 76 can collapse and, thus, reduce the cross-section of the distal end of the overtube device 68 in case no eleventh device 71 is provided in the distal opening. This is useful in particular when inserting the overtube device 68 into a tubular hollow organ since a small cross-section can allow an easy and gentle insertion. The lumen of the distal opening 76 can be extended by inserting an eleventh device 71. In one embodiment, the hose element 123 may include a closed cross-section connected to the distal end element 73 in a portion of the outer surface 124 of the hose element 123, as shown in FIG. 46a. In another embodiment, the hose element 123 may have an open cross-section connected to the distal end element 73 so that the distal opening 76 to be obtained has a closed cross-section, as shown in FIG. 46b.

In one embodiment, the tube element 72 is made entirely or partially of a flexible material, such as, for example, a plastic or synthetic film, which allows a collapsing of the lumen of the tube element 72 in case no eleventh device is provided in the tube element 72. By this, the cross-section of the overtube device 68 can be reduced. This may be useful, for example, when the overtube device 68 is inserted into a tubular hollow organ, since a small cross-section is adapted to allow an easy and gentle insertion of the overtube device 68. The lumen of the tube element 72 can be extended by an insertion of the eleventh device 71.

The flexibility of the overtube device 68 may be partially determined by the second control element 74. During insertion of the overtube device 68 into a hollow organ, a very high flexibility may be desired. In contrast thereto, when the distal end has reached the place of intervention, a low flexibility of the overtube device 68 may be desirable in order to reach high controllability of the distal end element 73.

In one embodiment, the second control element 74 may include a mechanism by which flexibility of the entire second control element 74 can be optionally adjusted.

In another embodiment, the second control element 74 may have a mechanism by which the flexibility of at least one portion of the second control element 74 can be optionally adjusted.

In still another embodiment, as shown in FIG. 47, the second control element 74 may have a control segment 125. The illustrated control segment 125 of the second control element 74 has a fourth control element 126 and a ninth operating element 127. The ninth operating element 127 may be located at the proximal end 70 of the overtube device 68 and may be connected to the control segment 125 via the fourth control element 126. The control segment 125 of the second control element 74 may be located at the distal end 69 of the overtube device 68.

The control segment 125 may be designed so that, upon actuation of the ninth operating element 127, the bending of the control segment 125 can be adjusted via the fourth control element 126. By adjustment of the bending of the control segment 125, the orientation of the distal end element 73 of the overtube device 68 can preferably be adjusted.

Furthermore, a stabilizing of the distal end 69 of the overtube device 68 may be desired, in particular when manipulating the target tissue by surgical instruments which are led out of the distal openings 76 of the overtube device 68. Such a stabilization can be effected by supporting the overtube device 68 on the hollow organ wall 129. In particular, in a tubular hollow organ with few variations in the cross-sectional area such as the large intestine, such a stabilization of the distal end 69 of the overtube device 68 can be effected.

In one embodiment, as shown in FIG. 49, the outer surface of the overtube device 68 may include at least one first fluid chamber 128. A fluid may be fed to the first fluid chamber 128 through one or more fluid feed devices 129 and discharged from the first fluid chamber 128 through one or more of the fluid feed devices 129. By feeding a fluid to the first fluid chamber 128, the cross-section of the overtube device 68 can be selectively extended.

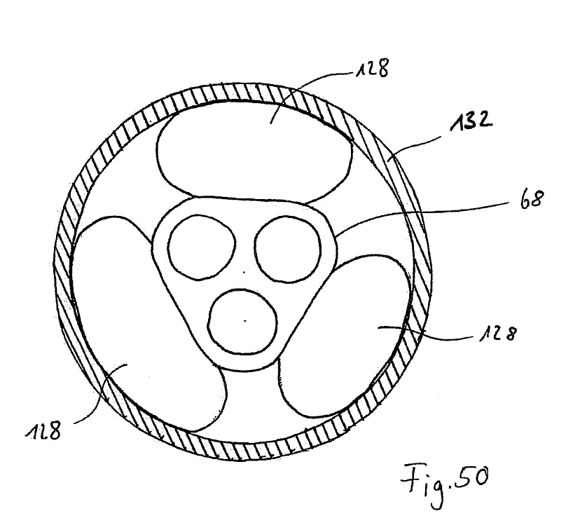

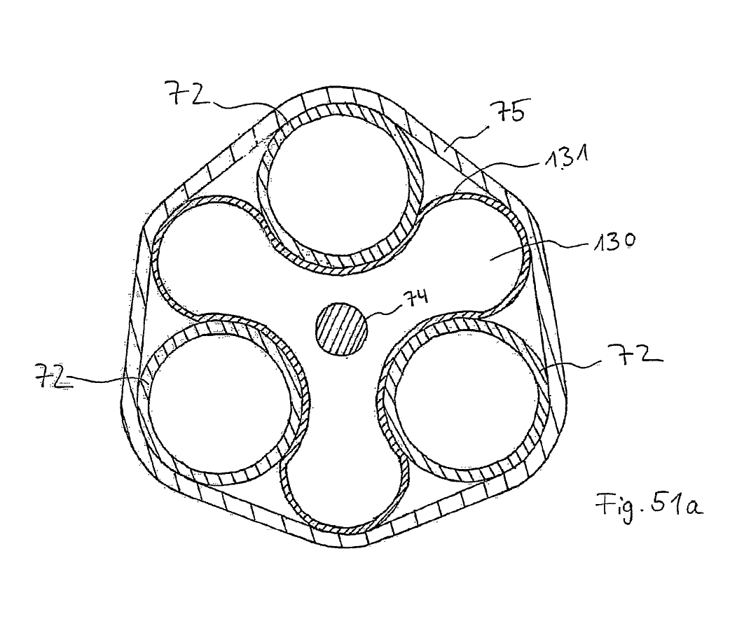

By feeding a fluid to the first fluid chamber 128, a stabilization of the distal end 69 of the overtube device 68 in a hollow organ can be achieved by supporting the overtube device 68 on the wall 129 of the hollow organ, as shown in FIG. 50. As shown in FIG. 48, the outer covering 75 may include at least one first fluid chamber 128.

According to an inventive aspect of the present application, the overtube device 68 may be provided with a collapsible structure. For this purpose, for example, the tube elements 72 and the outer covering 75 can be made of a flexible material, such as, for example, a plastic or synthetic film. In this way, the insertion of the overtube device 68 into the human body may be conducted more easily and more gently.

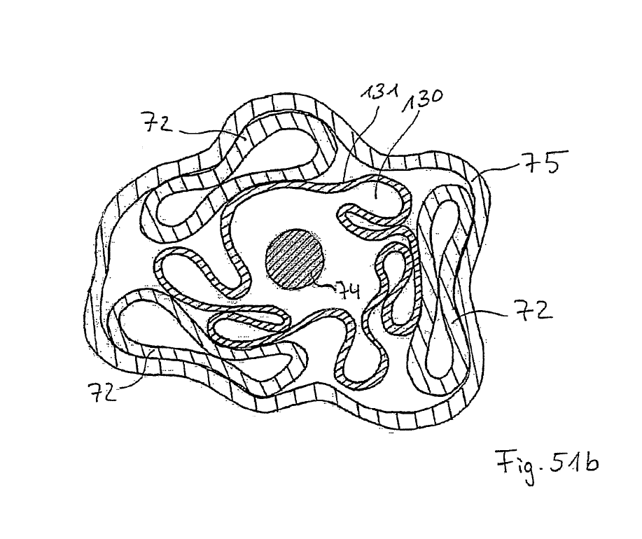

In another embodiment, as shown in FIGS. 51a and 51b, an overtube device 68 with a collapsible structure may include a fluid chamber system 131 including one second fluid chamber 130, which acts as a supporting structure when it is filled with fluid. This supporting structure can serve to re-establish the collapsed cross-section of the overtube device 68 and, for example, facilitate the insertion of an instrument 8 into the tube element 72. When the fluid chamber system 131 is insufficiently filled with fluid, the structure can collapse, as shown in FIG. 51b. If the fluid chamber system 131 is sufficiently filled with fluid, the expansion to a preferred cross-sectional shape of the overtube device 68 may be supported, as shown in FIG. 51a. The fluid may include any suitable fluids, including gases and/or liquids.

In another embodiment (not shown), a fluid chamber system 131, such as those described herein, may be divided in segments which are preferably arranged along the overtube device 68 at regular distances. Optionally, the segments of the fluid chamber system 131 may be selectively filled with fluid. As a result of the design of the fluid chamber system 131 including segments, a bending of the overtube device 68 can be maintained during filling a fluid into the fluid chamber system 131. The fluid may include any suitable fluids, including gases and/or liquids.

The overtube device 68 may have a symmetrical or an asymmetrical cross-section. For example, where the medical instruments to be inserted into the tube elements 72 have different diameters, an asymmetrical design of the cross-section of the overtube device by using tube elements 72 having different sizes may be desired.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

(1) Inner tube element

(2) Outer tube element

(3) First structures

(4) Tension element

(5) Shaft/instrument shaft

(6) Proximal end of the first device/proximal end of the instrument

(7) Distal end of the first device

(8) First device/instrument

(9) Axis of the first device/axis of the instrument

(10) Distal opening of the inner tube element

(11) Lateral recess

(12) Outside of the lateral recess

(13) Inside of the lateral recess

(14) Cutting surfaces of the clearance

(15) Front surface of the inner tube element

(16) Segment

(17) Hinge element

(18) Loop of the tension element

(19) Through bore of the segment

(20) Rotational axis of the hinge element

(21) Line tangential to the outer surface of the segment

(22) Axis of the segment

(23) Bending element

(24) Shaft of the inner tube element

(25) clearance

(26) Bending region

(27) Taper/narrowed portion

(28) Second tension element guide

(29) First mechanical connection

(30) Second mechanical connection

(31) Lateral opening in the outer tube element

(32) Proximal enlargement in the tension element

(33) Lateral opening in the inner tube element

(34) Distal enlargement in the tension element

(35) First tension element guide

(36) Guide member

(37) Groove in the inner surface of the inner tube element

(38) Through bore in the outer wall of the inner tube element

(39) Mechanical gear element

(40) Second lateral opening in the outer wall of the inner tube element

(41) Outer surface of the outer tube element

(42) Inner surface of the outer tube element

(43) Outer surface of the shaft of the inner tube element

(44) Inner surface of the shaft of the inner tube element

(45) Second device

(46) Fifth operating element of the second device

(47) Shaft of the second device

(48) Surgical effector

(49) Proximal end of the flexible surgical instrument

(50) Distal end of the flexible surgical instrument

(51) Outer surface of the second device

(52) Fourth device

(53) First control element

(54) First connecting element

(55) Fifth device/control device

(56) Sixth device

(57) Seventh device

(58) End element

(59) First operating element

(60) First element of the sixth device

(61) Second element of the sixth device

(62) Eighth device

(63) Second operating element

(64) Third element of the sixth device

(65) Ninth device

(66) Third operating element

(67) Axis of the sixth device

(68) Tenth device/overtube device

(69) Distal end of the tenth device

(70) Proximal end of the tenth device

(71) Eleventh device

(72) Tube element/instrument channel

(73) Distal end element

(74) Twelfth device/second control element

(75) Outer covering

(76) Distal openings

(77) Fourth operating element

(78) Axis of the tenth device

(79) Camera unit

(80) Instrument tip

(81) Preferred direction

(82) Distal end of the tension element

(83) Proximal end of the tension element

(84) Groove in the outer surface of the inner tube element

(85) Surgical instrument

(86) Surgical effector

(87) Second control element/force transmitting member

(88) First spur gear

(89) Second spur gear

(90) First bevel gear

(91) Second bevel gear

(92) Second connecting element

(93) Handle/grip

(94) Guiding element

(95) Turning device

(96) Second force transmitting member

(97) Preferred rotational direction

(98) Spring element

(99) Third connecting element

(100) Handle/grip

(101) Guiding element

(102) Deflection device

(103) Force transmitting member

(104) Turning device

(105) Preferred direction

(106) Deflection device

(107) Guiding means

(108) Spring element

(109) Fourth connecting element

(110) Handle/grip

(111) Seventh operating element

(112) Sixth operating element

(114) Inner surface of the tube element

(115) Outer surface of the tube element

(116) Outer surface of the eleventh device

(117) Guiding device

(118) Lateral opening of the guiding device

(119) Guiding segments

(120) Actuating device

(121) Third control element

(122) Eighth operating element

(123) Hose element

(124) Outer surface of the hose element

(125) Control segment of the second control element

(126) Fourth control element

(127) Ninth operating element

(128) First fluid chamber

(129) Wall of the hollow organ

(130) Second fluid chamber

(131) Fluid chamber system

We claim:

1. An insertion aid for medical instruments, comprising:
a shaft having a proximal end, a distal end, and a bending part having structures which allow a bending in at least one desired direction different from an extension direction of the shaft; and
a tension element for actuating the bending part;
wherein the shaft further comprises an outer tube element and an inner tube element supported in the outer tube element so as to be axially movable, wherein the tension element is hinged to one of the inner and outer tube elements and the bending part is provided at a distal end of the other of the inner and outer tube elements.

2. The insertion aid according to claim 1, wherein the bending part is arranged on the inner tube element, and the tension element is hinged to the outer tube element for actuating the bending part.

3. The insertion aid according to claim 1, wherein the bending part axially protrudes beyond the distal end of the outer tube element.

4. The insertion aid according to claim 1, wherein the structures of the bending part comprise a plurality of outer notches longitudinally spaced apart from each other in a wall of the one of the inner and outer tube elements.

5. The insertion aid according to claim 1, wherein the structures are formed by a plurality of segments longitudinally spaced apart from each other, the segments being connected to each other and to the one of the inner and outer tube elements by corresponding hinges to form a continuous chain of segments.

6. The insertion aid according to claim 5, wherein the tension element is connected to a distal end of the bending part in a decentralized maimer, the tension element being guided back through guiding bores provided in the segments and leading to the distal end of the other of the inner and outer tube elements for a transmission at least one of a tensile force and a compressive force to the distal end of the bending part.

7. The insertion aid according to claim 1, further comprising a handling device for manual relative displacement of the inner and outer tube elements to effect a bending of the bending part by a desired angle in accordance with the extent of the relative displacement.

8. The insertion aid according to claim 7, wherein one of the handling device and the shaft includes a locking device for maintaining a desired angular position of the bending part.

9. A handling device for actuating a bending part of an insertion aid comprising a shaft having a proximal end and a distal end, the bending part having structures which allow a bending in at least one desired direction different from an extension direction of the shaft, and a tension element for actuating the bending part, the shaft further comprises an outer tube element and an inner tube element supported in the outer tube element so as to be axially movable, wherein the tension element is hinged to one of the inner and outer tube elements and the bending part is provided at a distal end of the other of the inner and outer tube elements, the handling device comprising:
first and second trigger grips, wherein one end of each of the first and second trigger grips is connectable to the distal end of a corresponding one of the inner and outer tube elements for a relative displacement of the inner and outer tube elements in an axial direction.

10. The handling device according to claim 9, further comprising a locking device for locking a desired actuating position of the handling device to maintain a desired angular position of the bending part.

11. The handling device according to claim 9, wherein the handling device is configured to allow for a zero point adjustment for adjusting an initial relative position of the two tube elements in an initial position of the handling device.

12. A handling device for actuating a bending part of an insertion aid comprising a shaft having a proximal end and a distal end, the bending part having structures which allow a bending in at least one desired direction different from an extension direction of the shaft, and a tension element for actuating the bending part, the shaft further comprises an outer tube element and an inner tube element supported in the outer tube element so as to be axially movable, wherein the tension element is hinged to one of the inner and outer tube elements and the bending part is provided at a distal end of the other of the inner and outer tube elements, the handling device comprising:
a trigger grip connectable to a medical instrument assembled with the insertion aid for an axial displacement of the medical instrument.

13. The handling device according to claim 12, wherein the trigger grip is connectable to a medical instrument provided in the insertion aid.

14. The handling device according to claim 12, wherein the trigger grip is connectable to a medical instrument attached to the insertion aid.

15. The handling device according to claim 12, further comprising a locking device for locking a desired actuating position of the handling device to maintain a position of the medical instrument.

16. The handling device according to claim 12, wherein the handling device is configured to allow for a zero point adjustment for adjusting an initial position of the medical instrument in an initial position of the handling device.

17. A handling device for actuating a bending part of an insertion aid comprising a shaft having a proximal end and a distal end, the bending part having structures which allow a bending in at least one desired direction different from an extension direction of the shaft, and a tension element for actuating the bending part, the shaft further comprises an outer tube element and an inner tube element supported in the outer tube element so as to be axially movable, wherein the tension element is hinged to one of the inner and outer tube elements and the bending part is provided at a distal end of the other of the inner and outer tube elements, the handling device comprising:
a turning wheel connectable to a medical instrument assembled with the insertion aid for rotation of the medical instrument.

18. The handling device according to claim 17, wherein the turning wheel is connectable to a medical instrument provided in the insertion aid.

19. The handling device according to claim 17, wherein the turning wheel is connectable to a medical instrument attached to the insertion aid.

20. The handling device according to claim 17, further comprising a locking device for locking a desired actuating position of the handling device to maintain a desired orientation of the medical instrument.

21. The handling device according to claim 17, wherein the handling device is configured to allow for a zero point adjustment for adjusting an initial orientation of the medical instrument in an initial position of the handling device.

22. An overtube device for receiving one or more insertion aids each comprising a shaft having a proximal end and a distal end, a bending part having structures which allow a bending in at least one desired direction different from an extension direction of the shaft, and a tension element for actuating the bending part, the shaft further comprises an outer tube element and an inner tube element supported in the outer tube element so as to be axially movable, wherein the tension element is hinged to one of the inner and outer tube elements and the bending part is provided at a distal end of the other of the inner and outer tube elements, the overtube device comprising:
a hose-like tube element having one or more tube channels for sliding reception of at least one of an optical system and one or more of the insertion aids; and a distal end part connected to a torsion force transmission mechanism for twisting at least a distal end of the overtube device around a longitudinal axis of the overtube device.

23. The overtube device according to claim 22, wherein the torsion force transmission mechanism is coupled to at least one tube shaft so as to be axially movable.

24. The overtube device according to claim 22, further comprising a fluid chamber adapted to locally extend the cross-section of the overtube device when filled with a fluid.

25. The overtube device according to claim 22, wherein the tube channels are made of a deformable material to allow for a collapsing of the cross-section of the overtube device.

26. The overtube device according to claim 25, wherein the deformable material comprises a synthetic film.

27. The overtube device according to claim 25, further comprising a fluid chamber system configured to adjust a desired cross-sectional shape of the overtube device when filled with a fluid.
